(19) United States
(12) Patent Application Publication
(10) Pub. No.: US 2017/0088584 A1
(43) Pub. Date: Mar. 30, 2017

(54) USE OF PEPTIDIC DRUGS FOR OSTEOPOROSIS TREATMENT AND BONE REGENERATION

(71) Applicant: UNIVERSIDAD NACIONAL AUTONOMA DE MEXICO, Distrito Federal (MX)

(72) Inventors: Higinio ARZATE, Distrito Federal (MX); Fabiola SALGADO CHAVARRIA, Distrito Federal (MX); Gonzalo MONTOYA AYALA, Distrito Federal (MX)

(21) Appl. No.: 14/865,909

(22) Filed: Sep. 25, 2015

(51) Int. Cl. C07K 7/08; C07K 7/06

(52) U.S. Cl. CPC C07K 7/08; C07K 7/06; A61K 38/00

(57) ABSTRACT

The present invention provides osteogenic peptides derived from the Cementum-derived attachment protein (HACD1/CAP) and another derived from the Cementum Protein 1 (CEMP1) and pharmaceutical compositions of these peptides for the prevention and treatment of osteopenia and osteoporosis. These peptides increase bone mineral density in an osteoporotic model and without in vivo side effects, demonstrating clinical effectiveness in the prevention and treatment of osteopenia and osteoporosis in vivo as well as bone repair and/or regeneration.

### TECHNICAL FIELD OF THE INVENTION

The present invention relates to the field of medicine. New bio drugs characterized by their osteogenic properties are described, their applications and pharmaceutical compositions in the treatment of patients with diseases that affect bony structures such as osteopenia, osteoporosis are described and it also relates with its application in the area of Orthopedic Medicine and Dentistry to induce and/or increase bone formation and regeneration in a localized manner including the joint use of a tridimensional synthetic or natural scaffold.

### BACKGROUND OF THE INVENTION

Osteoporosis is a systemic disease of the skeleton characterized by low bone mass and micro architectural deterioration of the bone tissue, with the consequent increase in bone fragility and susceptibility to fracture (Eggertsen, 2007).

Osteoporosis is defined by the World Health Organization (WHO) as a bone mineral density of the hip or spine of 2.5 standard deviations below the normal figure determined in young people, usually expressed as a T marker of −2.5 or less (Eggertsen, 2007, Moayyeri, 2006, WHO, 2004).

Osteopenia or decrease in bone mass is defined as a bone mineral density between 1 and 2.5 standard deviations below the mean of normal individuals. (Eggertsen, 2007, Moayyeri, 2006, Sikon, 2006) It is of normal occurrence during aging and may or may not lead to osteoporosis. An individual is classified with severe osteoporosis when a fracture occurs due to inadequate bone mineral density. Osteoporosis is a relentless epidemic of rapid growth at global level. More than 200 million people in the world are affected by osteoporosis, of which 80% are women (NOF 2002). In the next decade and due to the growth of the adult population and therefore the potential increase in the number of patients with osteoporosis, it will be and it is necessary to establish therapeutics for the prevention and treatment of this public health problem worldwide. Likewise, Osteoporosis is associated with significant morbidity, mortality, and with a crushing economic burden for those who suffer from this disease.

The National Osteoporosis Foundation in the United States of America estimates that in this country osteoporosis is a serious health problem for 44 million people of 50 years of age and older, of whom 10 million have osteoporosis and 34 million have low bone mineral density (i.e., osteopenia) (NOF, 2002, Adler, 2006, Boonen, 2007). Of the ten million people with osteoporosis, 80% are women and 20% men (NOF 2002). However, according to the US Census Bureau of 2000, the number of individuals diagnosed with osteoporosis is likely to increase to 14 million men and women.

Similarly, the prevalence of osteoporotic fractures has increased dramatically in menopausal women (Cauley, 2006). Bone loss is more abrupt during the first decade after the establishment of the menopause. Expectations in this regard are not promising because 50% of women and 20% of men are expected to have a fracture in their life span. Therefore, the risks for women after a hip fracture equals the combined risk of breast cancer, uterine cancer and ovarian cancer and the risk of death due to a hip fracture is equal to the mortality of breast cancer. Currently the incidence of fractures exceeds 1.5 million per year. For the year 2025 it is predictable that there will be an increase in fractures of more than 3 million with an estimated cost of 25.3 billion dollars (Khosla, 2001). The model predicts a 2.7 times increase in fractures in population of Hispanic origin (A, 1998, CELADE, 2000, WBG, 2001, WHO, 2000). The spine fractures are the most common osteoporotic fractures and are associated with activities such as weight lifting. Vertebral fractures cause back pain, curvature of the spine, reduction in lung function, and increase in mortality (Delmas, 1997, Gehlbach, 2000, Klotzbuecher, 2000). Hip fractures are the second most common osteoporotic fracture and possess the highest morbidity, mortality, impact (damage) economic and subsequent loss of independence (Dennison, 2006, Johnell, 2005, Orwig, 2006). Also, due to the increase in life expectancy of the population, this type of fracture due to osteoporosis is increasing as a percentage. The third most common osteoporotic fracture is the distal bone of the forearm, followed by fractures of the humerus, other fractures of the femur, ribs, pelvic fractures, clavicle, scapula, and fractures of the sternum (breastbone), tibial and fibular fractures (Kaukonen, 1988, Klotzbuecher, 2000).

In Latin America, the decrease in bone mineral density and bone loss is similar in comparison to the United States of America. The prevalence of osteopenia in women aged 50 and older is 45.5% to 49.6% and vertebral osteoporosis is present in a range between 12.1% and 17.6%. In the neck of the femur, osteopenia is present in a range between 46% and 57% and osteoporosis, between 7.9% and 22% (Deleze, 2000, Santos, 2001, Lewin, 1996, Favus, 1993, Szejnfeld, 1995, Hui, 1997, ICSBM, 1997). In Mexico, one in every four people over the age of 50 suffers from osteopenia or osteoporosis, approximately 24.5 million people representing 22% of the population (Riera-Espinoza 2009). Since 1990 and projecting to the year 2050, the number of hip fractures in 50 to 64 year-old women and men will increase by 400%. For the age groups over 65 years, the increase will be up to 700% (Cooper C, 1992). Of all the people who suffer hip fracture in these age groups, 17% die 4 months after the fracture. Clinical studies have found that only 10% of the population over 70 years presented normal bone mineral density (Riera-Espinoza, 2003). In Mexico, 1 in every 12 women older than 50 years will suffer hip fracture. The percentages of mortality in Latin America related to osteoporotic fractures range from 1.02% to 10% during their stay in the hospital and 23% to 30% during the first year after the fracture. The percentage of mortality showed that it is higher in men than in women (Riera-Espinoza, 2003). The prevalence of vertebral fractures in women aged 50 and older is 19.35% and in women of 80 years of age and older, this percentage rises to 37%. The prevalence of vertebral fractures in Mexican men is half of that for Mexican women, 9.7% vs. 19.5%, with the highest percentage in men who are older than 80 years of age with 21.4% (Clark, P, 2006). This shows that the impact of osteoporotic fractures in Mexican men is equal to what is present in the rest of the world. These data support the evidence that with an increase in fracture incidence over the next 50 years, the magnitude of the health problem that osteoporotic fractures represent requires a comprehensive evaluation by the health systems in Latin American countries and in the rest of the world (Cooper C, 1992).

Osteoporosis develops less frequently in men than in women due to the fact that bone loss begins later and progresses more slowly in men, and neither does it experience a period of rapid hormonal changes that is also accompanied by rapid bone loss. The differences in bone geometry and remodeling also contribute to a lower percentage of fractures in men (Adler, R, 2006). However, in recent years, the problem of osteoporosis in men has been recognized as a major public health problem, particularly if you consider that the number of men older than 70 years will double between the years 1993 and 2050 according to the National Foundation for Osteoporosis of the United States of America. From this perspective 1 in 8 men over 50 years of age is afflicted by osteoporosis and currently 2 million men suffer from this disease and other 3 million are at risk (Burge, 2007). Each year men represent a third of all hip fractures and one third of these men will not survive more than a year and the damages caused by osteoporosis are severe and in some cases fatal. Currently, there are no clinical or chemical tests in blood or urine accurate enough to determine the abnormality that occurs during osteoporosis. At present the techniques generally used are: biochemical markers, radiographs and measurements of bone mineral density. However, the use of these techniques is limited for reasons of cost and accuracy (Allende-Vigo, 2007, Lane, 2006, Lata, 2007). The risk factors for menopausal women are predictive of fracture, independent of bone mineral density. Age is an independent risk factor for fractures; each decade of life in postmenopausal women increases 2 times the risk of fracture. Also, the continuous and intermittent use of high doses of steroids increases the risk of fracture. The combination of risk factors and determining bone mineral density enhances the possibilities to predict fractures.

In addition, laboratory tests must be guided by history and physical examination (Reddy, 2013). The chemical set of tests that may be useful includes: complete blood count, thyroid gland function, vitamin D 25-OH level, serum or urinary protein electrophoresis, level of parathyroid hormone and calcium levels in the urine of the last 24 hours (Allende-Vigo, 2007, Lane, 2006, Lata, 2007). Bone metabolism markers include formation and resorption bone markers, which are useful in some instances to monitor the course of therapy but not to be used for osteoporosis diagnosis. Currently the dual-energy X-ray absorptiometry (DEXA) is considered the gold standard for measuring bone mineral density and classifying osteoporosis (Blake, 2007, Holick, 2005, Miller, 2006). Non-pharmacological treatment of osteoporosis includes preventive measures such as visual correction and improvement of hearing impairment; exercise, which helps maintain bone mineral density in postmenopausal women (Miller2006, Bouxsein, 2007); nutritional supplement administration such as calcium and vitamin D, which are considered as fundamental before adding other pharmacological therapeutics as they have been shown to improve bone mineral density of the hip, however, they do not significantly reduce fractures (Parker, 2005, NAMS, 2006). It should be noted that calcium supplements frequently produce the following digestive adverse effects: constipation, flatulence and abdominal distention. They have also been linked with an increased risk of kidney stones formation. Vitamin D may cause alterations in plasma concentrations of calcium. An increase in the incidence of cardiovascular disease has been described in women who took supplements of 1.5 mg of calcium per day, however, these results should be interpreted with caution due to the methodological limitations of the study and because this adverse effect has not been evidenced in any other study with calcium.

The therapeutic value of calcium and vitamin D supplements has been scarcely analyzed. A study that assessed the compliance and persistence for different drugs in women with osteoporosis found that the lowest rate of compliance was for calcium and vitamin D supplements, since only half of the patients took more than 80% of the recommended doses. The intestinal absorption of calcium is very low, since it is estimated that only 30% of the total amount ingested is absorbed. Calcium supplements come in the form of various salts: carbonate, gluconate, lactate, citrate, etc. Although it has been described that there are differences in the absorption of the different calcium salts, they do not appear to have clinical significance and disappear when they are administered with food.

Pharmacological therapies are applied with the objective of improving bone mineral quality and reduce fracture risk. These drugs are grouped into two basic categories; anti-catabolic/anti-resorptive, which include hormone replacement therapy, estrogen agonists/antagonists, calcitonin, bisphosphonates, RANKL inhibitors (i.e., denosumab), and anabolic therapies such as the administration of Teriparatide (NAMS, 2006, Bonnick, 2006, Keen, 2007). Other promising therapies include the administration of strontium (Shoback, 2007). Hormone replacement therapy is an anti-catabolic treatment. Hormonal therapies that include a combination of estrogen and progesterone increase bone mineral density in post-menopausal women (McClung, 2006). In sum, estrogen plus progesterone significantly reduce vertebral, hip, forearm and wrist fractures. Hormone replacement relieves the symptoms of menopause such as vaginal dryness, hot flashes and reduces the risk of colon cancer (Cauley, 2006). However, the side effects include risk of developing breast cancer, venous thromboembolism, coronary artery disease and myocardial infarction. There is also concern about endometrial cancer in the uterus when estrogen therapy is not prescribed along with progesterone therapy. However, this estrogen/hormone therapy has been approved by the FDA for osteoporosis prevention, relief of hot flushes and vulvovaginal atrophy (Cauley, 2006). Raloxifene is a drug approved by the FDA which is an estrogen agonist/antagonist receptor and an anti-catabolic drug. Its administration increases bone mineral density in the lumbar spine, hip and neck of the femur. It also reduces the risk of vertebral fractures and recently Raloxifene received an additional approval by the FDA as a drug that decreases the risk of non-invasive breast cancer (Cauley, 2006, Harris, 2005). Its side effects include the risk of venous thromboembolism and hot flashes.

Calcitonin is a biological agent of the anti-catabolic kind and is a drug approved by the FDA for osteoporosis treatment. Its effects reflect that it is useful for slightly increasing bone mineral density in the spine and hip and it has been determined that its administration reduces vertebral fractures, but it was not concluded if calcitonin administration provides some benefit to reduce hip or vertebral fractures (Cummings, 1998). Because it is prescribed as a nasal spray, its side effects include nasal irritation and occasional bleeding. It also has a possible analgesic effect in patients with vertebral compression fractures (Chesnut, 2000). Bisphosphonates are pyrophosphate analogues and the anti-catabolic kind of drugs, they are used for osteoporosis treatment. Drugs that contain bisphosphonates and are approved by the FDA are used for the prevention and treatment of osteoporosis, osteoporosis related to glucocorticoids and osteoporosis in men. These drugs have been proven effective to increase bone mineral density in the hip and the spine and to reduce the number of spine, hip and vertebral fractures (Chesnut, 2008, Black, 1996). Side effects of these drugs include: gastrointestinal irritation and other potential collateral effects include: muscle-skeletal pain and mandibular osteonecrosis. Some reports have shown that continuous bisphosphonate treatment is important for the body's response to these drugs and therefore, for fracture reduction (Lieberman, 1995, Nevitt, 1993).

Teriparatide is a biological recombinant hormone indicated for the treatment of post-menopausal osteoporosis and osteoporosis in men. It is a drug consisting of the first 34 amino acids of parathyroid hormone. Teriparatide increases bone mineral density in the spine and hip and decreases vertebral non-vertebral fractures (Silverman, 2007). Its effectiveness has not been proven for the treatment of hip fractures. In studies in rats, the administration of this drug is associated with the presence of osteosarcoma. Its major side effects include: hypercalcemia, leg cramps and dizziness. Because of its association with the development of osteosarcoma, the FDA has limited its administration to 24 months.

RANKL inhibitors such as Denosumab are monoclonal antibodies produced against the receptor activator of nuclear kβ-ligand factor (RANKL). When Denosumab joins RANKL, it inhibits osteoclastic formation, function and survival, and for this reason, bone mineral density increases. It has been determined that Denosumab reduces vertebral, non-vertebral and hip fractures in post-menopausal women with osteoporosis (Neer, 2001). One of the potential side effects of Denosumab, is mandibular osteonecrosis, which shows a similar prevalence to that observed in patients using bisphosphonates.

The intact parathyroid hormone (PTH 1-84) and its analogue, teriparatide, represent a new kind of anabolic treatment for osteoporosis. A positive effect over bone microarchitecture improvement has been described as well as a reduction in the risk of new fractures due to a bone-forming mechanism. PTH 1-84 does reduce the risk of vertebral fracture but Teriparatide has only shown to decrease fracture risk but not specifically of vertebral fractures. PTH should be considered in the treatment of severe osteoporosis, both in men and women, in patients who have multiple osteoporotic fractures or very low bone mineral density (T-score less than −3.5) and a high risk for fracture. Other potential uses are in glucocorticoid-induced osteoporosis and other secondary osteoporosis. Teriparatide and PTH 1-84 use is not recommended for more than 24 months.

However, there is no evidence that PTH (1-84) possesses greater efficiency than the peptide 1-34. In fact the opposite seems to be more likely. Parathyroid hormone (peptide of 1-84) currently does not currently exist in the market in the USA or in Europe. Fragments of parathyroid hormone have been used as anabolic agents that stimulate bone formation. These fragments produce substantial increases in bone density and reductions in the risk of vertebral fractures and spinal cord. Currently its use is limited due to its high cost, the need for daily injections and concerns regarding the increase in osteosarcoma development based on animal studies. In any case, after a decade of clinical use there is no real evidence for this to be a concern in regard to its application in humans. Beneficial effects have also been shown additive to the use of teriparatide with zoledronate and denosumab.

Another alternative for osteoporosis treatment could be Bone Morphogenetic Proteins (BMPs). These are the most important family of growth factors for achieving bone regeneration and possess a wide range of biological activities which include: cartilage induction, bone formation, organogenesis, proliferation and apoptosis (Gautschi, 2007; Jansen, 2005). BMPs have been implemented in surgeries to recover damaged bone but the use of BMPs in food supplements to exert an effect over osteoporotic condition becomes complicated because the two main routes of administration, oral ingestion and topical application, do not ensure that the proteins will reach the bone. BMPs tend to break before reaching bone tissue and besides, it has also been seen that being soluble in water, they spread very fast in all body fluids. It has been determined that the administration of food supplements of hydrolyzed type I collagen together with BMPs, minerals and vitamins, may contribute to bone reconstruction, but BMPs must be supplied with some carrier that protects them from the environment of the stomach.

Through recombinant DNA technology, adequate amounts of recombinant morphogenetic human proteins (rhBMPs) have been obtained for research purposes. However, its use in clinical practice is not only costly, but it has also been associated with adverse effects such as bone overgrowth and immune responses of the host. Recently, it has been reported that short sequences of peptides from its central region could mimic the inductive properties of bone BMPs in terms of bone regeneration, suggesting that they can be used as an alternative inductor, (Jansen, 2005; Saito, 2004; Sheol, 2006; Lin XH, 2006). Bone morphogenetic proteins (BMPs) induce endochondral bone formation through stimulation of the differentiation process of mesenchymal progenitor cells (Hogan, 1996), and have been extensively assessed in a variety of applications which include treatment of spinal fusions, fractures, craniofacial and periodontal defects (King, 1997; Giannobile, 1998; Geesink, 1999; Cochran, 2000; Kirker-Head, 2000; Franceschi; 2005).

Only BMP-2 and BMP-7/OP1 recombinant human proteins have been approved by the FDA (Federal Drug Administration, USA) for clinical use in humans and it is limited only to orthopedics and spine fusion. Also, the administration of BMP-2 and BMP-7/OP1 significantly improves the initial states of cartilage repair. However, a larger dose or multiple applications is required in order to achieve the desired osteogenic effect. On the other hand, these proteins have a half-life of 1 to 4 hours, which means that a dose of exogenous application of BMP is equivalent to the endogenous amount present in 1000 human beings. The foregoing represents a serious concern regarding their safety and cost (Kwon 2005). Additionally, supra-physiological concentrations as a result of imperfect release kinetics of BMPs where 30% of the capsule is lost in the initial phase have been related to severe clinical complications including generalized hematomas in soft tissue and peri-implant bone resorption (Gautschi, 2003; Bessa, 2008). Another side-effect of the administration of BMP is that they promote excessive bone growth which results in pressure over the gastrointestinal tract and nerve roots, and they are powerful inducers of arterial calcification which may predispose the recipient individuals to serious complications such as thrombosis or atheroma.

As it has been mentioned, osteoporosis is characterized by a decrease in bone mass and micro-architectural deterioration of bone tissue with the consequent bone fragility and the resulting susceptibility to hip, spine and wrist fractures. This implies that the homeostatic balance between bone resorption and bone formation determines the mass and structural integrity of the skeleton, which is affected during the osteoporotic process. Some current therapeutic alternatives for osteoporosis treatment decrease the risk of osteoporotic fractures, reducing bone resorption and preserving its architecture but do not stimulate bone formation. Parathyroid hormone (PTH) and BMPs do stimulate bone formation but have the above-mentioned disadvantages. To elucidate the mechanisms that regulate bone formation can lead to the development of therapeutics that may be able to rebuild bone mass and its architecture. Currently, there is a need for more therapeutic options that provide better results to treat this disease.

Cementum Proteins

Cementum is a mineralized connective tissue that covers the roots of teeth and possesses unique characteristics. The physical-chemical and structural characteristics of the cementum are well known and have been described in detail to a morphological level. It has been shown that cementum contains polypeptides with unique location in the cementum and cementoblasts, these polypeptides include the cementum-derived attachment protein (HACD1/CAP) and the cementum protein 1 (CEMP1). They play an important role in cell recruitment and differentiation during the formation of radicular cementum (Alvarez, 2006).

Recently we cloned and identified mRNA from a library derived from a cell line of cemento-ossifying human fibroma which encodes a truncated isoform of the 3-hidroxiacil-coenzyme A-dehydratase 1 (HACD1); its homonym is CAP. CAP gene encodes for a 140 amino acid protein of which 125 amino acids of the Terminal-N are identical to HACD1, which encodes for 288 amino acids. The rest of the carboxyl terminal of CAP is the splicing donor site in the exon 2 of HACD1. CAP has two transmembrane sites and the protein is truncated after the second transmembrane site. This truncation eliminates the HACD1 sequence that consists of the following amino acids: IVHCLIGIVPT (SEQ ID NO. 3); which is the reason for a phosphatase active site that is replaced by the 15-aminoacid sequence VSFPSCCFSIAVIFM (SEQ ID NO. 4). Therefore, CAP is an isoform, and alternative splicing and homonym of HACD1 (Valdes, 2012).

As mentioned above, CAP encodes for a product of 140 amino acids and alternative splicing occurs exactly at the active site of HACD1, by what we infer that its role may be different. This alternative splicing includes the carboxyl terminal of the VSFPSCCFSIAVIFM (SEQ ID NO. 4) protein. This sequence is unique in the databases. More importantly, the peptide that has been synthesized has demonstrated unique properties of biological utility because it has high affinity for hydroxyapatite and also promotes hydroxyapatite crystals formation in vitro and their supramolecular organization.

Cementum protein 1 (CEMP1), is a protein of 247 amino acids with a Mᵣ of 50,000 Daltons and several posttranslational modifications of glycosylation and phosphorylation; it is expressed by cell subpopulations of the human periodontal ligament, cementoblasts that surround the cementum, stem cells of perivascular location in the periodontal ligament and pericytes in the alveolar bone, cartilage of the spinal cord and in the upper and lower extremities in during development. CEMP1 presents no homology to any other protein only with amino acids 30 to 110 it presents a 48% similarity with αI chain collagen type I, 46% with type XI collagen and 40% with type X collagen.

The patent application that has been submitted to the Mexican Institute of Intellectual Property Protection in May 7, 2012; number of request: MX/a/2012/005314; refers to the peptide VSFPSCCFSIAVIFM (SEQ ID NO. 4) in tissue engineering for bone regeneration where there has been a severe loss of bone tissue, thus making possible de novo bone formation with this technology.

The Mexican patent No. 324953 with date of request Dec. 8, 2009 refers to obtaining pharmaceutical formulations that comprise recombinant Cementum protein 1 (CEMP1), which induces cementum, bone, dentin, periodontal ligament and cartilage formation and which is combined with polylactic co-glycolic acid (PLGA) that facilitates the release of CEMP1 protein.

Currently, techniques such as molecular biology and genetic engineering are used for the production of recombinant proteins, however, the complexity of the equipment, as well as the preparation techniques, long production cycles, low yields and costly processes make it difficult to achieve a large-scale production. The main differences in the bio-pharmaceuticals produced by methods of recombinant gene expression in relation to those produced by chemical synthesis consist in the greater molecular size and the complexity of its molecular structure. Due to their nature, they have a low oral bioavailability, not exceeding 1% most of the times. This is mainly due to enzyme activity in the digestive tract, where a high metabolism of proteins and peptides is produced, and on the other hand, because of the barrier function produced to avoid the absorption of these molecules in the intestinal wall. Also, tissue distribution is complex, being continuously exposed to degradation by the action of proteolytic enzymes present in blood; in general, the half-life is usually small, which is why the protein structure of some bio drugs is joint with polymers (polyethylene glycol-PEG, for example) to avoid rapid renal excretion.

In addition to this, there are security problems of products as a result of genetic engineering (Wozney, 2004). Many bio drugs are capable of causing the generation of neutralizing antibodies, which could reduce or even eliminate their impacts on their drug targets. The majority of the bio-pharmaceuticals have been classified as hospital medicines, in many cases, this condition is determined by the need to perform a close monitoring in patients during or after the administration; in other cases such justification is questionable, given that some of these medications are specifically equipped for self-administration by the patient.

To date, there are no drugs that meet the requirements of a material with no side effects used for osteoporosis treatment. Osteoporosis is a pathological entity that cannot be cured in short term, since it requires administration of drugs over a long period of time.

According to the latter, there is a need for a different drug or bio drug that does not display any of the side effects mentioned above and that is therapeutically effective for the replacement of conventional drugs, which is synthesis of easy and finally that is economically accessible to the population for the attention of this public health problem in the world.

Considering the abovementioned, the present invention relates to the use of synthetic peptides derived from the Cementum Attachment Protein (HACD1/CAP) and also of a peptide derived from the Cementum Protein 1 (CEMP1), which promotes an increase in bone mineral density and a restoration of bone microarchitecture as well as a restoration of the bone's physical-mechanical properties and biological properties in subjects with osteoporosis. Specifically, the present invention relates to the selection and characterization of peptides, one from HACD1/CAP protein and located in the terminal carboxyl and whose sequence is AVIFM (SEQ ID NO: 1) and the other peptide derived from the cementum protein 1 (CEMP1) of the amino terminal sequence: MGTSSTDSQQAQHRRCSTSN (SEQ ID NO: 2). We refer to their uses and formulations which contain them in a pharmaceutically accepted soluble vehicle to promote osteogenesis; they are supplied from the profile of biological activities in order to counteract the problems posed along the description.

In the state of the art these peptides or their properties have not been characterized nor described previously, in this request we describe its properties and characterization, which makes them excellent candidates for the prevention and treatment of osteopenia, osteoporosis, and osteogenesis promotion to maintain bone homeostasis.

### BRIEF DESCRIPTION OF THE FIGURES

FIG. 1: Hydroxyapatite crystals induced in a cell-free system in presence of AVIFM (SEQ ID NO: 1) peptide (20 μg/mL), in metasilicate gels. (A) Microspheres of homogeneous quadrangular and flat hydroxyapatite crystals. B) In the center of the microsphere a solid core with hydroxyapatite crystals in the form of radiating needles is observed. (C) Formation of structures using bovine serum albumin (20 μg/mL), these plates do not correspond to hydroxyapatite crystals. (D) Formation of structures without adding to the system any protein at all, these plates do not correspond to hydroxyapatite crystals. (E) Supramolecular organization and assembly of the hydroxyapatite crystals as revealed by means of atomic force microscopy. (F) High resolution transmission electron microscopy (HRTEM) of hydroxyapatite crystals.

FIG. 2: hydroxyapatite crystals induced in a cell-free system in the presence of peptide MGTSSTDSQQAQHRRCSTSN (SEQ ID NO: 2) (20 μg/mL) in metasilicate gels. Panel (A) microspheres of homogeneous quadrangular and flat hydroxyapatite crystals. Panel (B) in the center of the microsphere a solid core with hydroxyapatite crystals in the form of radiating needles is observed. Panel (C) formation of structures using bovine serum albumin (20 μg/mL), these plates do not correspond to hydroxyapatite crystals. Panel (D) formation of structures without adding to the system any protein at all, these plates do not correspond to hydroxyapatite crystals. (E) Supramolecular organization and assembly of the hydroxyapatite crystals as revealed by means of atomic force micros supramolecular organization and assembly of the hydroxyapatite crystals as revealed by means of atomic force microscopy. (F) High resolution transmission electron microscopy (HRTEM) of hydroxyapatite crystals.

FIG. 3: different spectra of Energy Dispersive X-rays (EDS) with their respective Ca/P ratios and where the peaks corresponding to the calcium and phosphorus may be observed: A) EDS of the crystals formed in the presence of peptide AVIFM (SEQ ID NO: 1) (20 μg/mL). The Ca/P ratio: 1.53, corresponds to hydroxyapatite of the biological kind. B) EDS of the crystals in presence of bovine serum albumin (20 μg/mL). The Ca/P ratio: 1.16, does not correspond to hydroxyapatite. C) EDS of the crystals formed in the sample without addition of bovine serum albumin or any peptide. The Ca/P ratio: 1.13, shows that these crystals do not correspond to hydroxyapatite.

FIG. 4: different spectra of Energy Dispersive X-rays (EDS) with their respective Ca/P ratios and where the peaks corresponding to the calcium and phosphorus may be observed: A) EDS of the crystals formed in the presence of peptide MGTSSTDSQQAQHRRCSTSN (SEQ ID NO: 2) (20 μg/mL). The ratio Ca/P 1.7 corresponds to hydroxyapatite of the biological kind. B) EDS of the crystals in presence of bovine serum albumin (20 μg/mL). The Ca/P ratio: 1.2, does not correspond to hydroxyapatite. C) EDS of the crystals formed in the sample without the addition of bovine serum albumin or any peptide. The Ca/P ratio: 1.1, tells us that these crystals do not correspond to hydroxyapatite.

FIG. 5: Microphotographs of 3 histological sections of the epiphyses of femur of Wistar rats. (A): control without treatment, histologic features of normal healthy bone. (B): ovariectomized rat with subsequent development of osteoporosis after ninety days and administration of physiological saline solution. (C): ovariectomized rat with the subsequent development of osteoporosis, ninety days after ovariectomy, received treatment with the peptide AVIFM (SEQ ID NO: 1) at a dose of 20-50 μg/kg of body weight in physiological saline solution by intraperitoneal route.

FIG. 6: Microphotographs of 3 histological sections of the epiphyses of femur of Wistar rats. (A): control without treatment, histologic features of normal healthy bone. (B): ovariectomized rat with subsequent development of osteoporosis after ninety days and administration of physiological saline solution. (C): ovarioectomized rat with the subsequent development of osteoporosis, ninety days after ovariectomy, received treatment with the peptide MGTSSTDSQQAQHRRCSTSN (SEQ ID NO: 2) at a dose of 20-50 μg/kg of body weight in physiological saline solution by intraperitoneal route.

FIG. 7: Microphotographs of 6 histological sections of the body of the 5^{th} lumbar vertebra in the transverse and sagittal planes. (A): Corresponds to the image of the 5^{th} lumbar vertebra in the transverse (a) and sagittal (a′) planes of normal Wistar rats. (B): Corresponds to the 5^{th} lumbar vertebra in the transverse (b) and sagittal (b′) planes of osteoporotic Wistar rat treated with saline solution. Panel (C), corresponds to the 5^{th} lumbar vertebra in the transverse (c) and sagittal (c′) planes of Wistar rats treated with the peptide AVIFM (SEQ ID NO: 1) at a dose of 20-50 μg/kg of body weight for 90 days of daily administration by intraperitoneal route.

FIG. 8: Microphotographs of 6 histological sections of the body of the 5^{th} lumbar vertebra in the transverse and sagittal planes. (A): corresponds to the image of the 5^{th} lumbar vertebra in the transverse (a) and sagittal (a′) planes of normal Wistar rats. (B): corresponds to the 5^{th} lumbar vertebra in the transverse (b) and sagittal (b′) planes of osteoporotic Wistar rat treated with saline solution. Panel (C), corresponds to the 5^{th} lumbar vertebra in the transverse (c) and sagittal (c′) planes of Wistar rats treated with the peptide MGTSSTDSQQAQHRRCSTSN (SEQ ID NO: 2) at a dose of 20-50 μg/kg of body weight for 90 days of daily administration by intraperitoneal route.

FIG. 9: Microphotographs of histological sections of tissues from normal Wistar rats and ovariectomized Wistar rats that developed osteoporosis. (A) Tissues in healthy Wistar rats without osteoporosis: (B) tissue of Wistar rats that developed osteoporosis and received the peptide AVIFM (SEQ ID NO: 1) at a dose of 20-50 μg/kg of body weight in physiological saline solution by intraperitoneal route for 90 days. L=liver, K=kidney, H=heart, Lu=lung, B=brain, S=Spleen, M=muscle. There are no histological characteristics suggesting that the administration of peptides in physiological saline solution causes microscopic alteration in these tissues.

FIG. 10: Microphotographs of histological sections of tissues from normal Wistar rats and ovariectomized Wistar rats that developed osteoporosis. (A) Tissues in health status of Wistar rats without osteoporosis: (B) tissue of Wistar rats that developed osteoporosis and received the peptide MGTSSTDSQQAQHRRCSTSN (SEQ ID NO: 2) at a dose of 20-50 μg/kg of body weight in physiological saline solution by intraperitoneal route and for 90 days. L=liver, K=kidney, H=heart, Lu=lung, B=brain, S=Spleen, M=muscle. There is no histological feature that indicates that peptide administration in physiological saline solution causes microscopic alteration in these tissues.

FIG. 11: Computed micro tomography images (μCT) of the vertebral body of the 5^{th} lumbar vertebra of Wistar rats (A) group not subjected to ovariectomy and that received no treatment at all; (B): group that underwent ovariectomy and did not receive treatment; (C): group that underwent ovariectomy and 5 months after, treatment was initiated with the peptide derived from cementum adhesion protein (HACD1/CAP): AVIFM, at a concentration of 20-50 μg/Kg weight dissolved in physiological saline solution and injected intraperitoneally daily during 90 days; computed micro tomography images μCT) of the vertebral body the 5^{th} lumbar vertebra of Wistar rats (D) group not subjected to ovariectomy and that Received no treatment at all; (E): group that underwent ovariectomy and did not receive treatment; (F): group that underwent ovariectomy and 5 months after, treatment was initiated with the peptide derived from cementum protein 1 (CEMP1): MGTSSTDSQQAQHRRCSTSN (SEQ ID NO: 2), at a concentration of 20-50 μg/Kg weight dissolved in physiological saline solution and injected intraperitoneally daily during 90 days. The inset within the figure shows with increase for each image.

FIG. 12: Resistance to compression of the body of the 5^{th} lumbar vertebra of normal Wistar rats (control), with osteoporosis (OVX) and with osteoporosis that received treatment with any of the peptides of the invention. (A): AVIFM (SEQ ID NO: 1). (B): MGTSSTDSQQAQHRRCSTSN (SEQ ID NO: 2); at a dose of 20-50 μg/kg of body weight and 90 days of daily administration intraperitoneally. The measurements were made at the end of treatment.

FIG. 13: Alkaline phosphatase specific activity in serum extracted from normal Wistar rats (control), with osteoporosis (OVX) and with osteoporosis that received treatment with any of the peptides: (A): AVIFM (SEQ ID NO: 1), (B): MGTSSTDSQQAQHRRCSTSN (SEQ ID NO: 2); at a dose of 20-50 μg/kg of body weight and for 90 days of daily administration by intraperitoneal route. The alkaline phosphatase specific activity was determined at the beginning and at the end of treatment.

FIG. 14: Circulating osteocalcin levels in serum extracted from Wistar rats, determined by ELISA, at the beginning and the end of the experimental period which lasted 90 days after a period of 5 months following ovariectomy. Serum from 8 healthy Wistar rats (control), 8 ovariectomized Wistar rats treated with intraperitoneal injection of physiological saline solution during 90 days (OVX) and (A): 8 osteoporotic ovariectomized Wistar rats treated with the peptide AVIFM (SEQ ID NO: 1), or (B) treated with the peptide MGTSSTDSQQAQHRRCSTSN (SEQ ID NO: 2); both at a concentration of 20-50 μg/Kg weight dissolved in physiological saline solution, and injected intraperitoneally, daily for 90 days 5 months after ovariectomy.

FIG. 15: Concentration of the receptor-activator for nuclear factor κ B ligand (RANKL) circulating in the serum extracted from Wistar rats at the beginning and the end of the 90 day experimental period 5 months after ovariectomy. The levels of RANKL were determined by means of an ELISA assay in serum obtained from 8 healthy Wistar rats (control), 8 ovariectomized Wistar rats treated with intraperitoneal injection of physiological saline solution during 90 days (OVX) and 8 osteoporotic ovariectomized Wistar rats treated with any of the peptides object of this invention (A): AVIFM (SEQ ID NO: 1), or (B): MGTSSTDSQQAQHRRCSTSN (SEQ ID NO: 2); at a concentration of 20-50 μg/Kg weight dissolved in physiological saline solution, and injected intraperitoneally daily for 90 days 5 months after ovariectomy.

FIG. 16: Circulating Osteoprotegerin level (OPG) in serum extracted from Wistar rats at the beginning and at the end of the 90-day experimental period 5 months after ovariectomy. The levels of osteoprotegerin were determined by means of an ELISA assay in sera obtained from 8 healthy Wistar rats (control), 8 ovariectomized Wistar rats and treated with intraperitoneal injection of physiological saline solution during 90 days (OVX) and 8 osteoporotic ovariectomized Wistar rats treated with any of the peptides, (A): AVIFM (SEQ ID NO: 1), or (B): MGTSSTDSQQAQHRRCSTSN (SEQ ID NO: 2); at a concentration of 20-50 μg/Kg weight dissolved in physiological saline solution injected intraperitoneally daily for 90 days 5 months after ovariectomy.

FIG. 17: Biodistribution of the AVIFM (SEQ ID NO: 1) peptide marked with pertechnectate (TcO₄_{^{−}}), levels detected in different tissues and blood of Wistar rats.

FIG. 18: Biodistribution of the MGTSSTDSQQAQHRRCSTSN (SEQ ID NO: 2) peptide marked with pertechnectate (TcO₄_{^{−}}), levels detected in different tissues and blood of Wistar rats.

FIG. 19: Cytotoxicity and proliferation in vitro using the peptides that are object of this invention (A): AVIFM (SEQ ID NO: 1) or (B): MGTSSTDSQQAQHRRCSTSN (SEQ ID NO: 2) in increasing concentrations: 1, 3 and 5 μg/ml.

FIG. 20: Microphotograph of the histologic section with a 5 μm thickness that shows the critical-size calvarial defect in Wistar rats. (A) No treatment, after 16 weeks, (B) the critical size defect was covered with a gelatin-based three-dimensional scaffold, after 16 weeks, (C) treatment with the three-dimensional scaffold formulation+20-50 μg of the peptide AVIFM (SEQ ID NO: 1) after 16 weeks.

FIG. 21: Microphotograph of the histologic section with a thickness of 5 μm that shows the critical-size calvarial defect in Wistar rats. (A) No treatment, after 16 weeks, (B) the critical size defect was covered with a gelatin-based three-dimensional scaffold, after 16 weeks, (C) treatment with the three-dimensional scaffold formulation+20-50 μg of the peptide MGTSSTDSQQAQHRRCSTSN (SEQ ID NO: 2) after 16 weeks

### DETAILED DESCRIPTION OF THE INVENTION

Bone mineral tissue deposition and bone tissue regeneration is required to cure certain diseases, and/or osteopenia sequel, osteoporosis, osteoarthritis. To correct the anomalies that these pathological entities cause over the bony structures, drug therapy to prevent and regenerate the bony structures is required. The present invention provides peptides synthesized artificially which promote the synthesis of hydroxyapatite and generate osteoinductive signals by what it constitutes an alternative of one of the key elements to aid bone mineral tissue deposition and bone tissue regeneration and in general for the development of technologies in the field of bone mineral tissue engineering.

The invention provides peptides that possess defined biological activity; these peptides are fragments of protein sequences derived from the cementum: cementum-derived attachment protein (HACD1/CAP) and cementum protein 1 (CEMP1). These peptides consist of sequences of 5 and 20 amino acids defined as SEQ ID NO:1 and SEQ ID NO:2 in the sequence listing respectively and may be used with an inert diluting agent such as a pH 7.4 physiological saline solution in a pharmaceutical formulation to promote the osteogenesis process and inhibit the process of bone resorption in vivo.

The present invention provides pharmaceutical compositions that contain either one of these peptides or both, which may be administered prophylactically and/or as inducer, and/or as a promoter with therapeutic utility with the purpose of increasing bone mineral density, restoring bone microarchitecture as well as the physical-mechanical and biological properties of bone tissue that are affected during osteopenia and osteoporosis and also to promote in vivo bone regeneration.

This invention is advantageous because these peptides prevent mineral bone density loss and its side effects such as vertebral, hip, head of the femur fractures and others associated with osteoporosis, both in men and women. The therapeutic usefulness of these peptides is related to the bone remineralization process so that it happens faster and without adverse side effects; which ultimately may improve the rate of clinical effectiveness and positively impact treatment cost. These peptides are then very useful for the prophylaxis and treatment of osteopenia and osteoporosis and for bone regeneration. In this invention the specific formula is described, as well as the peptide sequence, preparation methods and uses that occur within the scope of this invention.

The peptides related to the present invention have been synthesized by conventional biochemical methods. The synthesis of these peptides keeps the biological activity properties of the native protein and of human recombinant proteins; cementum-derived attachment protein (HACD1/CAP) and cementum protein 1 (CEMP1), so that the peptides role is similar to that of the cementum-derived attachment protein (HACD1/CAP), cementum protein 1 (CEMP1), and to the recombinant proteins HACD1/CAP and CEMP1. The present invention provides an alternative for increasing mineral bone density; promote bone mineral tissue deposition and bone neoformation and/or regeneration.

The ability of the peptide of the invention to inhibit the process of bone resorption, to promote osteogenesis, including the deposition bone mineral tissue, the regeneration and/or repair of normal bone conditions that have been lost due to osteoporosis, has been defined experimentally. Tests include in vitro results and experimental studies in an in vivo osteoporotic model in Wistar rats as well as bone regeneration in critical-size calvarial defects in preclinical studies in calvarias of Wistar rats.

As already mentioned, the structure of the biologically active peptides derived from the cementum-derived attachment protein (HACD1/CAP) are: AVIFM (SEQ ID NO: 1), or from cementum protein 1 (CEMP1); MGTSSTDSQQAQHRRCSTSN (SEQ ID NO: 2). These peptides were used in a pharmaceutical formulation with an injectable saline solution vehicle so that this combination is the preferred mode of the invention.

The pharmaceutical formulation disclosed in the present invention may increase bone mineral density, promote mineral bone tissue deposition and bone tissue regeneration that has been damaged as a result of a systemic disease such as osteoporosis; restore qualities related to bone size, morphology and microarchitecture as well as material and biomechanical properties of the bone. It also shows that these peptides are able to regulate supramolecular organization of nanospheres that constitute hydroxyapatite crystals and therefore act as initiators and regulators of the bio mineralization process. In addition it is shown that these peptides regulate serum levels of the biochemical markers involved in the biological process of resorption and/or deposition of extracellular matrix components of bone and bone mineral deposition.

Synthetic peptides with a purity of 95% that represents the carboxyl terminal of the human cementum-derived attachment protein (HACD1/CAP) with the sequence AVIFM (SEQ ID NO: 1), or the sequence representing the amino terminal of cementum protein 1 (CEMP1), MGTSSTDSQQAQHRRCSTSN (SEQ ID NO: 2), are synthesized by the conventional Fmoc/TBU method in solid-phase synthesis.

In order to obtain benefits from the use of peptides derived from the cementum-derived attachment protein (HACD1/CAP): AVIFM (SEQ ID NO: 1), and from cementum protein 1 (CEMP1); MGTSSTDSQQAQHRRCSTSN (SEQ ID NO: 2), synthesized artificially, the pharmaceutical formulations mentioned above were prepared with the objective of administering them properly. This pharmaceutical formulation is within the modalities of the invention, but does not limit the scope of the use that may be given to this peptide. The formulation consists in the dissolution of the peptide to a concentration of 20-50 μg/kg of body weight in physiological saline solution as described in various formulations of therapeutic products approved by the US Food and Drug Administration (FDA). The objective of this formulation is to facilitate the release of the invention peptides for their therapeutic utility or as a regulator and modifier of the therapeutic response in the induction of bone mineral tissue deposition and bone tissue regeneration in skeletal systemic anomalies characterized by low bone mass and damage of bone tissue microarchitecture as it so happens in osteopenia and osteoporosis.

As demonstrated experimentally and described below, these peptides significantly stimulate bone matrix deposition and guide the natural process of biomineralization. The peptides can facilitate self-assembly of nanospheres and nucleation of hydroxyapatite and enable them to grow in a structure similar to the one that occurs in natural bone, by which the peptides object of this invention can function in a similar manner to a native or recombinant protein and regulate the expression of molecules associated with the process of bone resorption/regeneration as is the case of alkaline phosphatase (ALP), osteocalcin (OCN), osteoprotegerin (OPG) and the receptor activator for nuclear factor κ B ligand (RANKL). All of the above mentioned provide as a result an increase in bone mineral density that is reflected in a mechanical resistance to compression with similar values to that of healthy bone.

In the present invention, the peptides used were artificially synthesized and combined with physiological saline solution at a concentration of 20-50 μg/kg of body weight. An experimental induction model for hydroxyapatite crystal formation was carried out in vitro and an experimental model of osteoporosis in Wistar rats was performed along with treatment of the latter with either one of the peptides described in this invention. Based on the experiments described below, we demonstrated that the peptides object of this invention may be used individually for the treatment of osteoporosis as described in example 1.

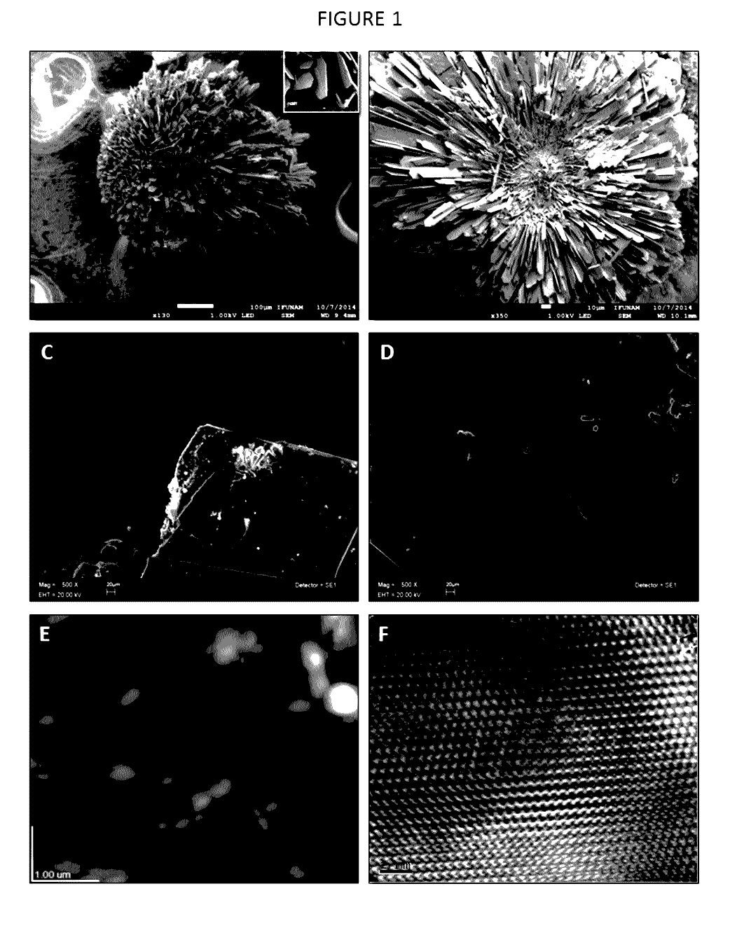

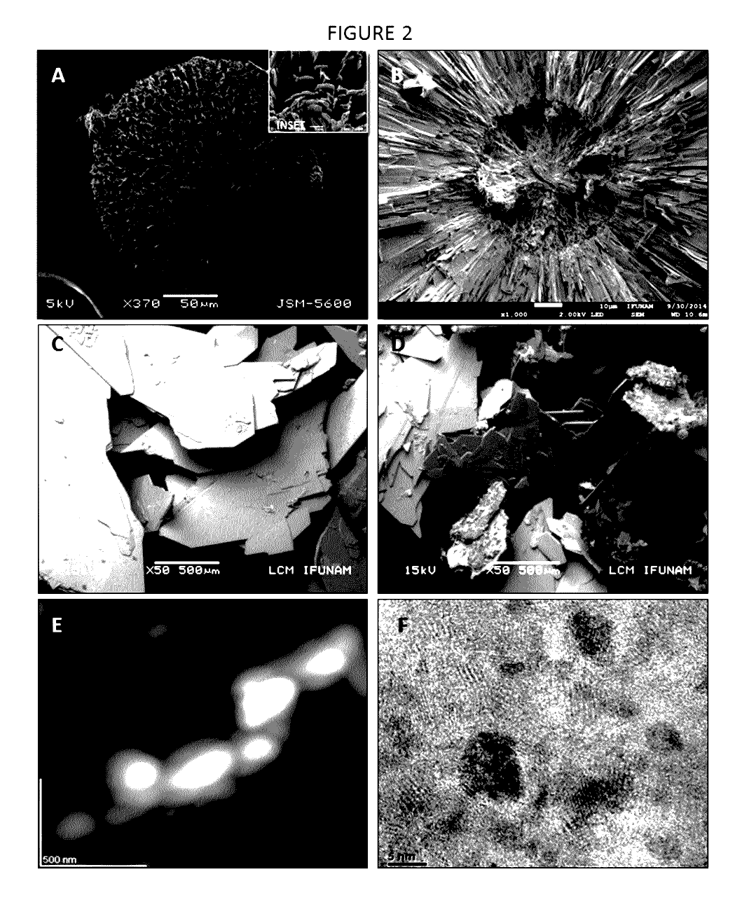

The obtained results are shown in FIGS. 1 and 2 where it may be may be observed the hydroxyapatite crystal induction in a cell-free in vitro system by means of either one of the peptides object of this invention and that correspond to the following sequences of the cementum-derived attachment protein (HACD1/CAP): AVIFM (SEQ ID NO: 1) or of the cementum protein 1(CEMP1): MGTSSTDSQQAQHRRCSTSN (SEQ ID NO: 2); used at concentration of 20 μg/mL in metasilicate gels, where the peptides were incubated separately for 7 days at a temperature of 37° C. and a concentration of 20 μg/mL in semi-solid gel of sodium metasilicate.

As observed in FIGS. 1(A) and 2(A) either one of the peptides promotes the formation of microspheres composed of homogenous crystals with an arrow-like ending and a beveled termination that correspond to hydroxyapatite crystals.

In FIGS. 1(B) and 2(B) the inner portion of a microsphere with its nuclei may be observed and from where crystals irradiate needle-like in their first portion and along their length they form flat crystals that correspond to hydroxyapatite crystals.

In FIGS. 1(C) and 2(C) crystal formation in a flat shape and with a plaque-like appearance may be observed with the use of a control protein which in this case was bovine albumin serum at a 20 μg/mL concentration. This plaque-shaped crystals do not correspond to hydroxyapatite.

In FIGS. 1(D) and 2(D) crystal formation in a flat shape and a plaque-like appearance may be observed, however in this control no protein was used and crystals do not correspond with hydroxyapatite. In FIGS. 1 (E) and 2 (E) a hierarchic organization may be observed as well as the self-assembly of nanospheres that arrange themselves in chains due to the alignment and fusion of the original nanospheres. In FIGS. 1(F) and 2(F) hydroxyapatite crystal analysis may be observed by means of high resolution electron microscopy (HRTEM) where the interplanar distance close to the planes 115 and 225 in the direction of the hydroxyapatite crystalline phase is shown.

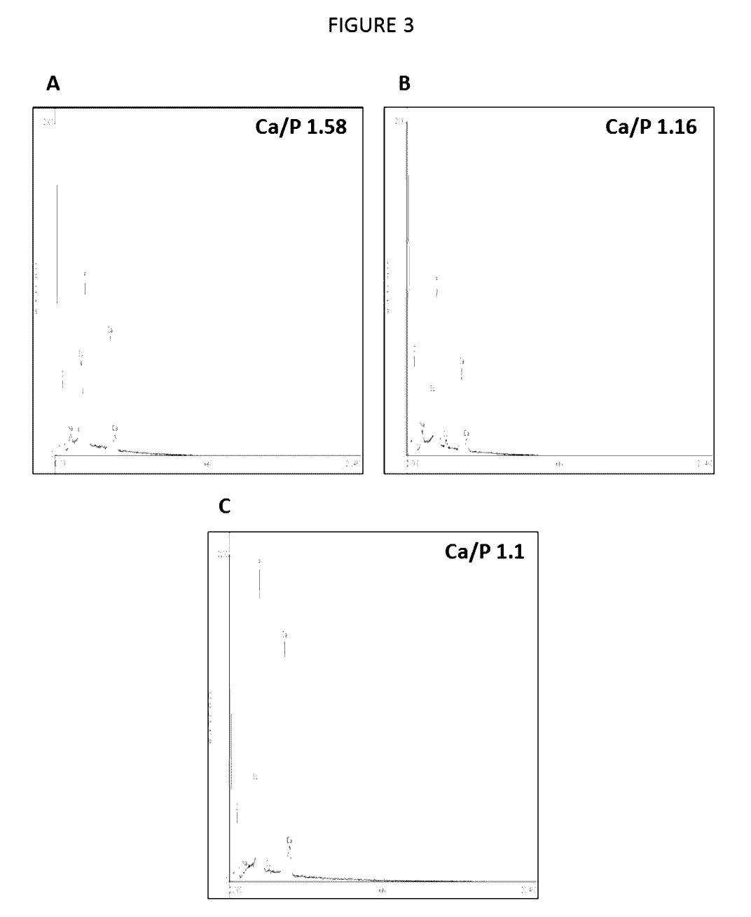

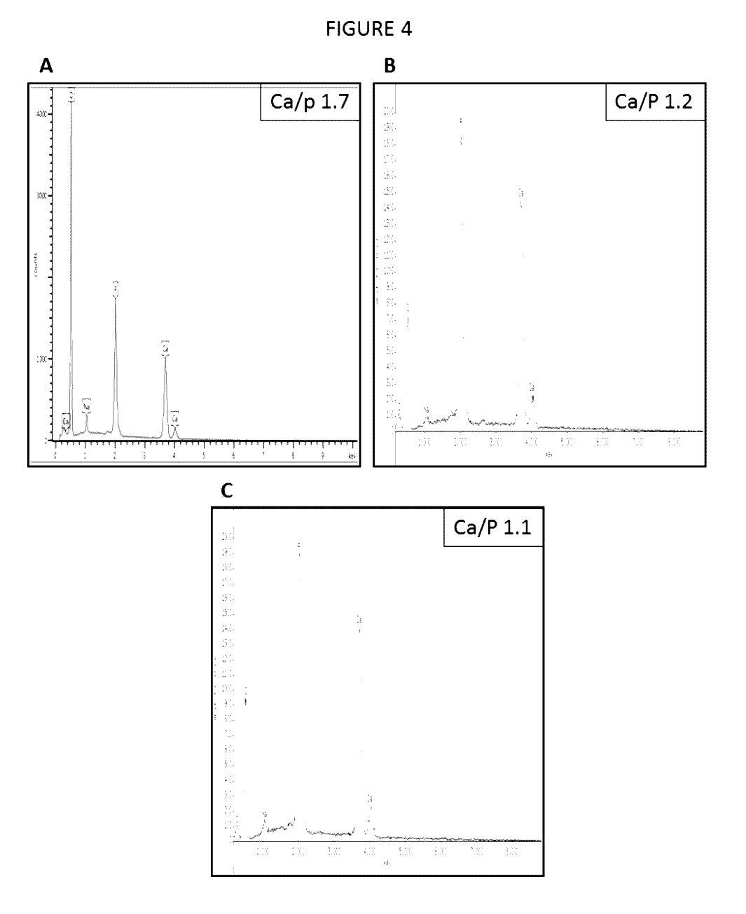

FIGS. 3 and 4 show the different spectra of Energy Dispersive X-ray (EDS) to determine crystal composition formed by the action of the previously mentioned peptides with their respective Ca/P ratio and where the corresponding peaks of calcium and phosphorus. Likewise the control spectra are shown with bovine albumin serum (20 μg/mL). The Ca/P ratio 1.16 to 1.2 shown in Panel B of FIGS. 3 and 4 does not correspond to hydroxyapatite. The Ca/P ratio according to the obtained spectra from the crystals induced by each one of the peptides object of this invention were found in a Ca/P ratio of 1.5 to 1.7 which are within the range of hydroxyapatite of the biological kind (Joint Committee on Powder Diffraction Standards No. 9-432 file for calcium hydroxyapatite) (JCPDS, 1998). This may be observed in Panel A of FIGS. 3 and 4 for each peptide (HACD1/CAP): AVIFM (SEQ ID NO: 1) or Cementum Protein 1(CEMP1): MGTSSTDSQQAQHRRCSTSN (SEQ ID NO: 2). In Panel C of FIGS. 3 and 4 the crystals formed without the presence of any protein may be observed with a Ca/P ratio that equals 1.1 and does not correspond to hydroxyapatite.

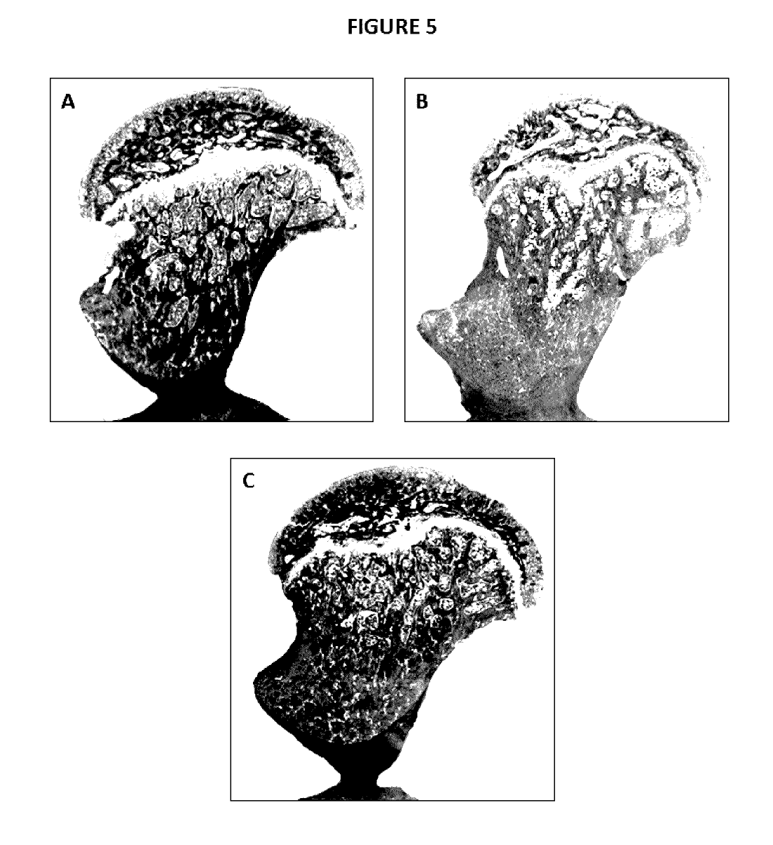

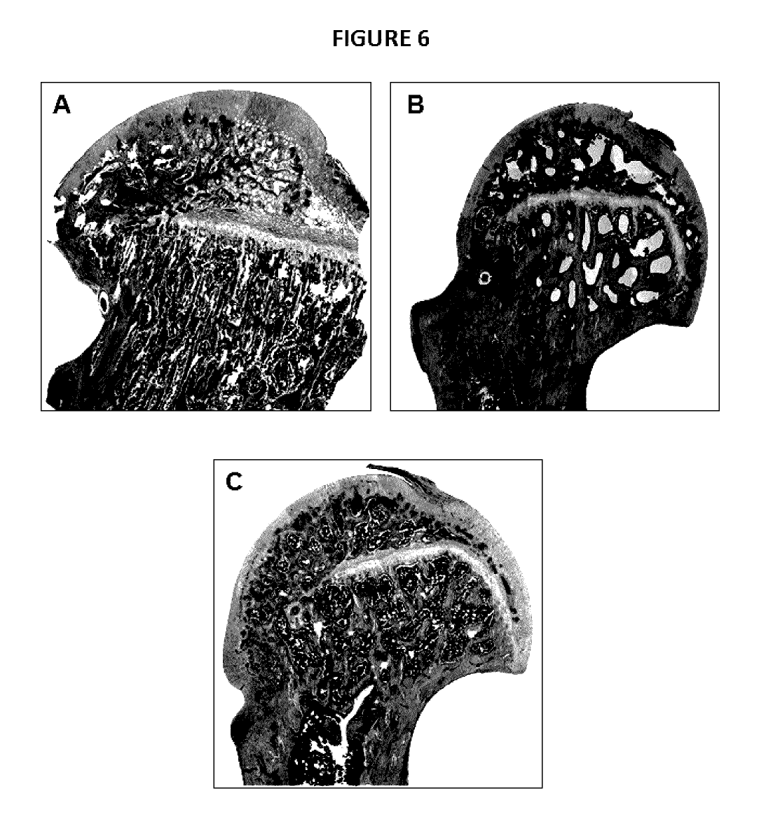

In FIGS. 5 and 6 histological sections of 5 μm thick and stained with Masson's Trichromic stain may be observed in which the microarchitecture of the epiphysis of the femur of the Wistar rats is analyzed in detail. Letter (A) of both figures corresponds to the group with no treatment which is the healthy control group. In the histological section stained with Masson's Trichromic stain, normal microarchitecture of the femoral epiphysis is observed with the cancellous bone and medullar spaces filled with bone marrow and bone occupying inter-medullar spaces. The epiphyseal plate and articular cartilage are also observed. Letter (B) of both figures shows the microarchitecture of the femur epiphysis of Wistar rats and corresponds to the group that underwent ovariectomy, developed osteoporosis and received as treatment the vehicle injection (physiological saline solution) 5 months after the ovariectomy. Treatment with the vehicle lasted 90 days. Tissue analysis at the end of the experimental period with physiological saline solution reveals in the microphotograph histological characteristics of the femur epiphysis of Wistar rats. Microarchitecture is also observed, where the medullar spaces of the epiphysis do not contain bone marrow, they are empty so the bone looks porous and with a sponge-like appearance. These spaces have variable sizes. Articular cartilage and epiphyseal plate are also noted. Letter (C) of both figures shows the microarchitecture of the femur epiphysis of Wistar rats that corresponds with the group subjected to ovariectomy that developed osteoporosis and received as treatment the pharmaceutical formulation with any of the peptides derived from the cementum-derived attachment protein (HACD1/CAP): AVIFM (SEQ ID NO: 1) or cementum protein 1 (CEMP1): MGTSSTDSQQAQHRRCSTSN (SEQ ID NO: 2); at a 20-50 μg/Kg of body weight, dissolved in saline solution and injected intraperitoneally daily for 90 days 5 months after the ovariectomy was performed. Microphotography showing the microarchitecture of the femur epiphysis of Wistar rats. This histological section stained with Masson's Trichromic stain shows the normal microarchitecture of the femur epiphysis with cancellous bone and where medullar spaces are filled with bone marrow and bone occupying intermedullar spaces. Articular cartilage and epiphyseal plate are also observed. This micrograph with histological characteristics shows that the Wistar rats that underwent ovariectomy, developed osteoporosis and were treated with the peptide object of this invention exhibit normal healthy bone characteristics.

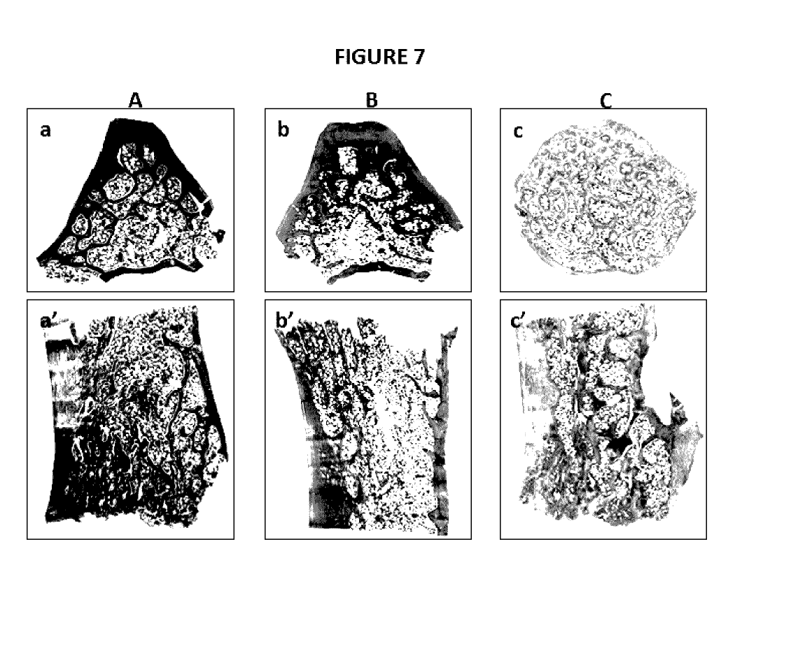

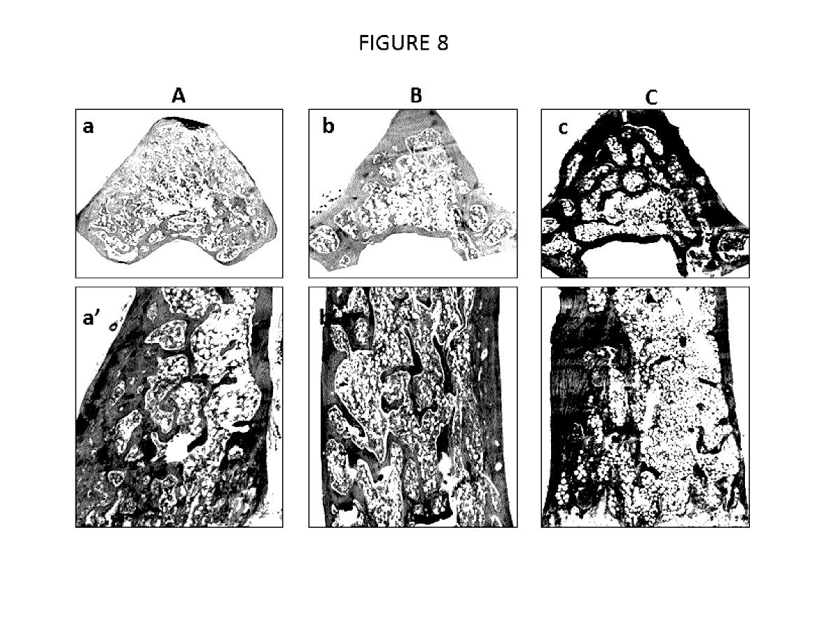

In FIGS. 7 and 8, microphotographs of histological sections of 5 μm thick of the 5^{th} lumbar vertebra in the transverse and sagittal plane stained with Masson's Trichromic stain. Panel (A) of FIGS. 7 and 8 corresponds to the body of the 5^{th} lumbar vertebra in the transverse (a) and sagittal (a′) planes of normal Wistar rats where cortical bone, bone trabeculate and spaces filled with bone marrow may be observed. Panel (B) of FIGS. 7 and 8 corresponds to the body of the 5^{th} lumbar vertebra in the transverse (b) and sagittal (b′) planes of osteoporotic Wistar rats treated with physiological saline solution where cortical bone and bone trabeculate are observed with lesser width than the one of healthy Wistar rats. Medullar spaces are also observed and they are filled with fatty tissue with larger areas than those found in healthy controls.

Panel (C) of FIGS. 7 and 8 corresponds to the body of the 5^{th} lumbar vertebra in the transverse (c) and sagittal (c′) planes of Wistar rats treated, respectively, with the peptide AVIFM (SEQ ID NO: 1) or the peptide MGTSSTDSQQAQHRRCSTSN (SEQ ID NO: 2) at a 20-50 μg/Kg of body weight administered daily by intraperitoneal route for 90 days where cortical bone is observed similar to that of healthy Wistar rats and a dense, well-organized trabeculate bone with medullar spaces filled with bone marrow is also observed.

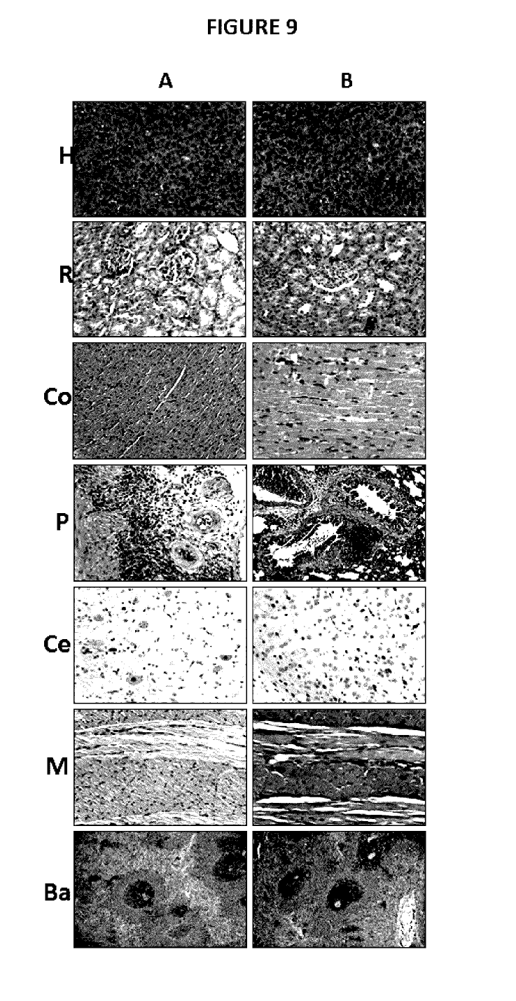

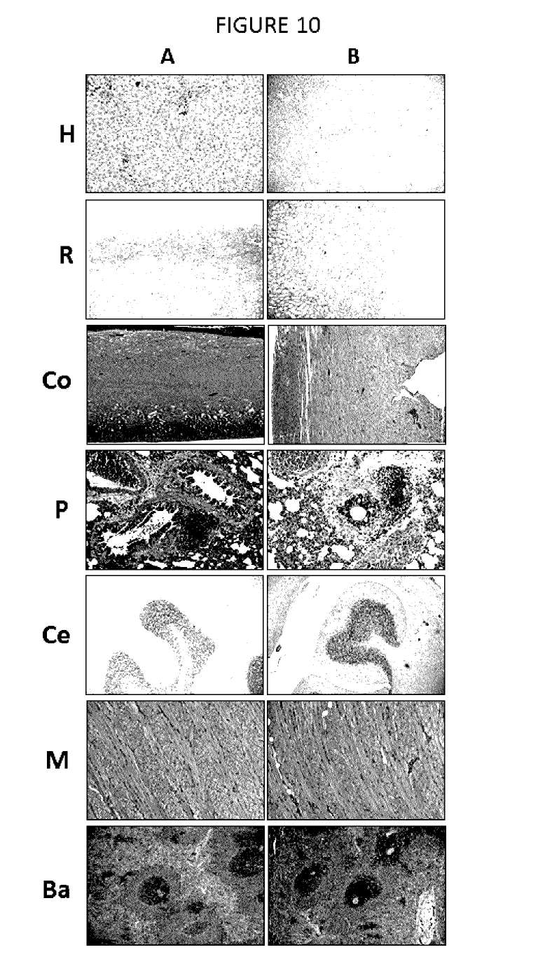

FIGS. 9 and 10 are microphotographs of histological sections of 5 μm thick that show histological characteristics of several tissues of Wistar rats that do not present osteoporosis and Wistar rats that underwent ovariectomy, developed osteoporosis and were treated with any of the synthetic peptides object of this invention derived from Cementum-Derived Attachment Protein (HACD1/CAP): AVIFM (SEQ ID NO: 1) or from Cementum Protein 1 (CEMP1): MGTSSTDSQQAQHRRCSTSN (SEQ ID NO: 2); at a concentration of 20-50 μg/Kg of weight dissolved in physiological saline solution and injected intraperitoneally daily for 90 days 5 months after the ovariectomy was performed.

The control tissues in Panel (A) correspond to: normal tissues: L=liver, K=kidney, H=heart, Lu=lung, B=brain, M=muscle, S=spleen. Panel B) tissues of Wistar rats with osteoporosis treated with the pharmaceutical formulations object of this invention; L=liver, K=kidney, H=heart, Lu=lung, B=brain, S=Spleen M=muscle. There were no differences in the microscopic tissue, nor alterations in their microarchitecture therefore, the tissues treated experimentally with the peptides object of this invention do not cause cellular or tissue damage, nor morphological alterations, thus normal characteristics, similar to those of healthy subjects are observed. This means that the synthetic peptides are harmless drugs for osteoporosis treatment, producing no adverse or secondary reactions at cellular or tissue level.

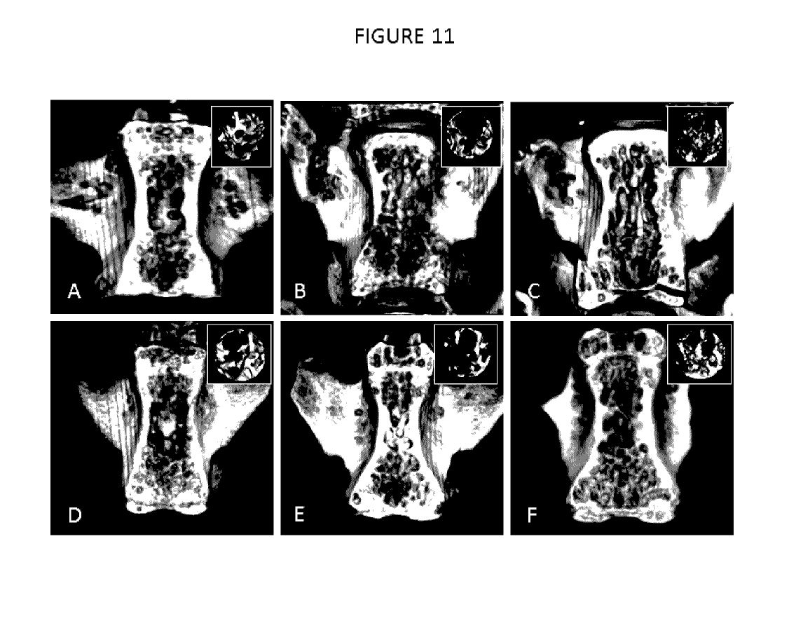

FIG. 11 presents images acquired through computed micro tomography (μCT) and three-dimensional reconstruction of the vertebral body of the 5^{th} lumbar vertebra of Wistar rats that were not subjected to ovariectomy and therefore represent the healthy control group, the group that underwent ovariectomy and was not treated with any of the peptides object of this invention and the group that underwent ovariectomy and that 5 months after, treatment was begun with any of the synthetic peptides object of this invention derived from the Cementum-Derived Attachment Protein (HACD1/CAP): AVIFM (SEQ ID NO: 1) or the Cementum Protein 1 (CEMP1): MGTSSTDSQQAQHRRCSTSN (SEQ ID NO: 2); at a concentration of 20-50 μg/Kg of weight, dissolved in physiological saline solution and injected intraperitoneally daily for 90 days, 5 months after ovariectomy.

Group that received treatment with the peptide AVIFM (SEQ ID NO: 1) and its controls: Panel A) the upper-lower image of the 5^{th} lumbar vertebra of the healthy control group is observed, particularly the vertebral body with all its anatomical components including the transverse apophysis, spinous process; the foramen where we see the bone cortex as a compact line as well as cancellous bone occupying all the space of this anatomic entity without any spaces. This is corroborated in the insert where it may be observed in detail the trabecular structures and marrow spaces. Panel B) image obtained through computed micro tomography of the 5^{th} lumbar vertebra of Wistar rats subjected to ovariectomy that developed osteoporosis and received as treatment the vehicle of this pharmaceutical formulations consisting of physiological saline solution injected intraperitoneally for 90 days. Anatomical vertebral features in an upper-lower position are observed and described in Panel (A). Three-dimensional reconstruction shows that the vertebral body presents medullar spaces of greater size and decrease of the bone trabeculae as well as of its thickness. In the inserted image cancellous bone may be observed in detail, with larger spaces and areas where there is a clear absence of cancellous bone. Panel C) image obtained through μCT of the 5^{th} lumbar vertebra of Wistar rats subjected to ovariectomy that developed osteoporosis and that received as treatment the pharmaceutical formulations consisting of either peptide of the object of this invention derived from the Cementum-derived Attachment Protein (HACD1/CAP): AVIFM (SEQ ID NO: 1) at a concentration of 20-50 μg/Kgn of weight dissolved in physiological saline solution and injected intraperitoneally daily for 90 days, 5 months after the ovariectomy. In the three dimensional reconstruction, the upper-lower image of the 5^{th} lumbar vertebra with all its anatomical components is observed. The three dimensional image shows us the electron-dense cortical of the vertebral body and cancellous bone with small spaces where cancellous bone is present in almost the entire vertebral body.

Group that received treatment with the MGTSSTDSQQAQHRRCSTSN (SEQ ID NO: 2) peptide and its controls: Panel D) shows the upper-lower image of the 5^{th} lumbar vertebra of the healthy control group, particularly the vertebral body with all its anatomical components including the transverse apophysis, spinous process; the foramen where we see the bone cortex as a compact line as well as cancellous bone occupying all the space of this anatomic entity without any spaces. This is corroborated in the insert where it may be observed in detail the trabecular structures and marrow spaces. Panel E) image obtained through computed micro tomography of the 5^{th} lumbar vertebra of Wistar rats subjected to ovariectomy that developed osteoporosis and received as treatment the vehicle of this pharmaceutical formulations consisting of physiological saline solution injected intraperitoneally for 90 days. Normal anatomical vertebral features in an upper-lower position as described in panel (A). Three-dimensional reconstruction shows that the vertebral body presents larger medullar spaces and a decrease of bone trabeculae as well as of its thickness. In the inserted image it may be observed in detail the spongy bone with larger spaces and areas where there is a clear absence of cancellous bone. Panel F) image obtained through computed micro tomography of the 5^{th} lumbar vertebra of Wistar rats subjected to ovariectomy that developed osteoporosis and received as treatment the pharmaceutical formulations consisting of either peptide of the object of this invention derived from the Cementum Protein 1 (CEMP1) at a concentration of 20-50 μg/Kg of weight dissolved in physiological saline solution, and injected intraperitoneally daily for 90 days 5 months after the ovariectomy. In the three dimensional reconstruction, the upper-lower image of the 5^{th} lumbar vertebra with all its anatomical components may be observed. The three dimensional image shows us the electron-dense cortical of the vertebral body and cancellous bone with small spaces where cancellous bone is present in almost the entire vertebral body.

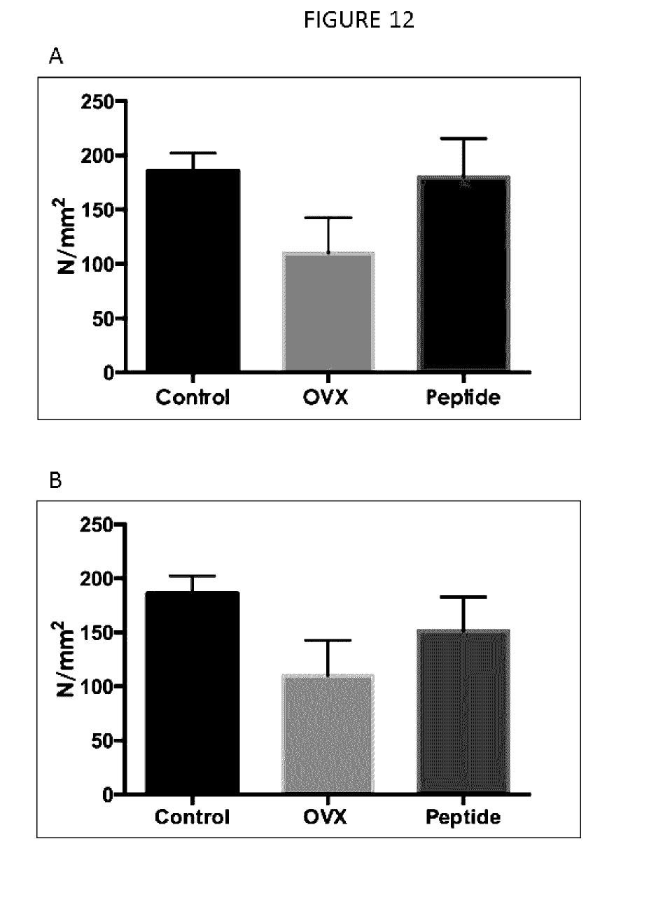

FIG. 12 corresponds to a graph showing the resistance to compression of the vertebral body of the 5^{th} lumbar vertebra obtained from Wistar rats at the end of the 90-day experimental period, 5 months after ovariectomy. Resistance Tests were performed using an Inströn Universal Testing Machine (mod. 5567) in the body of the 5^{th} lumbar vertebra compressed at a speed of 1 mm/min up to achieve sample fracture. The exerted force is expressed in N/mm². We analyzed the vertebral bodies of the 5^{th} lumbar vertebra of 8 healthy Wistar rats, 8 ovariectomized Wistar rats treated with intraperitoneal injection of physiological saline solution for 90 days and 8 osteoporotic ovariectomized Wistar rats treated with any of the peptides object of this invention derived from the Cementum-derived Attachment Protein (HACD1/CAP): AVIFM (SEQ ID NO: 1) or from Cementum Protein 1 (CEMP1): MGTSSTDSQQAQHRRCSTSN (SEQ ID NO: 2) at a concentration of 20-50 μg/Kg of weight dissolved in physiological saline solution, injected intraperitoneally daily, for 90 days 5 months after the ovariectomy was performed. As can be seen in the graph in FIG. 12, the resistance levels of the vertebral bodies of the 5^{th} lumbar vertebra of the osteoporotic rats (OVX) presented a 53% decrease in the resistance to compression. Osteoporotic rats treated with the peptide AVIFM (SEQ ID NO: 1) exhibit similar resistance to compression values as those of healthy control rats, exhibiting a 2% difference compared to control. As seen in graph (B), FIG. 12, the resistance levels of the vertebral bodies of the 5^{th} lumbar vertebra of osteoporotic rats (OVA) presented a 58% decrease in resistance to compression. Osteoporotic rats treated with the peptide MGTSSTDSQQAQHRRCSTSN (SEQ ID NO: 2) object of this invention exhibit similar resistance to compression values as those of healthy control rats, exhibiting a 19% difference compared to control. These results indicate that osteoporosis treatment with any of the peptides object of this invention promote resistance to compression and resistance to fracture similar to healthy control bone, suggesting that any of these peptides promote an increase in bone mineral density

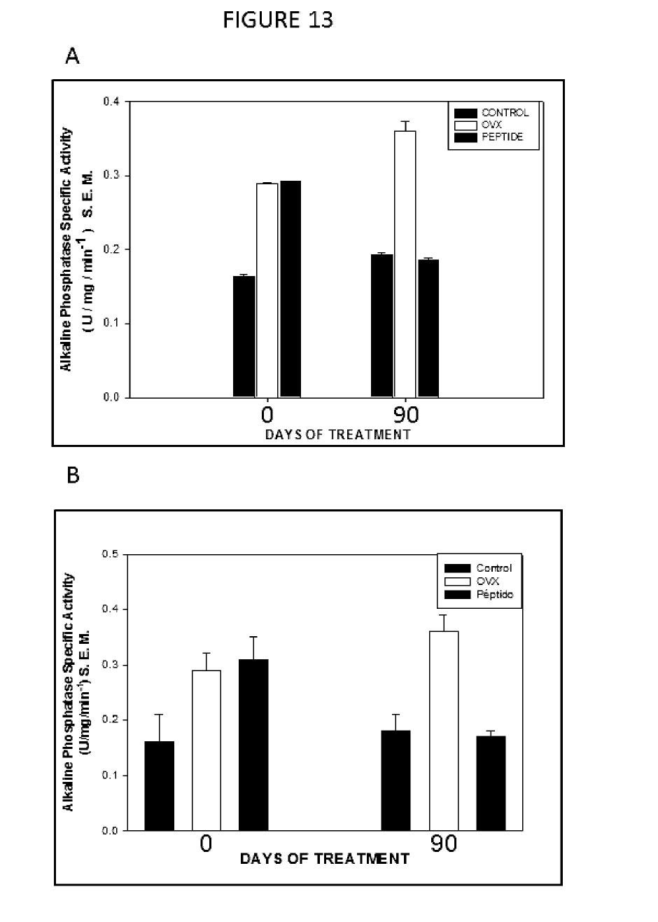

FIG. 13 corresponds to graphics of the specific activity of alkaline phosphatase in serum extracted from Wistar rats at the beginning and end of the 90-day experimental period 5 months after ovariectomy. We analyzed the specific activity of alkaline phosphatase from the serum of 8 healthy Wistar rats, 8 ovariectomized Wistar rats treated with intraperitoneal injection of physiological saline solution for 90 days and 8 osteoporotic ovariectomized Wistar rats treated with any of the peptides object of this invention derived from the Cementum-derived Attachment Protein (HACD1/CAP): AVIFM (SEQ ID NO: 1) (FIG. 13A) or Cementum Protein 1 (CEMP1): MGTSSTDSQQAQHRRCSTSN (SEQ ID NO: 2) (FIG. 13B); at a concentration of 20-50 μg/Kg of weight dissolved in physiological saline solution and injected intraperitoneally daily for 90 days 5 months after ovariectomy. As noted in the chart, at the beginning of treatment osteoporotic rats show an increase of 53% of the specific activity of serum alkaline phosphatase with respect to the values of the same enzyme in healthy, normal rats. After 90 days, at the end of treatment, the osteoporotic Wistar rats treated with any of the peptides object of this invention show a difference of 1% compared to healthy control Wistar rats and a decrease of 48% regarding the specific activity of alkaline phosphatase which remains high in osteoporotic rats treated with the vehicle used in this pharmaceutical formulation (physiologic saline solution). These results indicate that the peptides object of this invention are able to regulate the specific activity of alkaline phosphatase and therefore the peptide participates in the bone remineralization process in osteoporosis treatment.

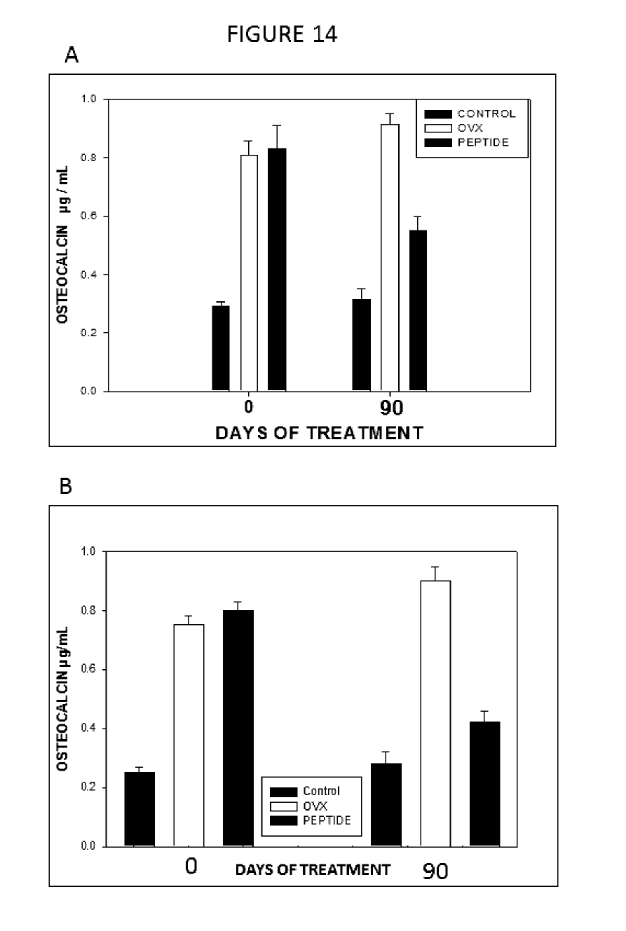

FIG. 14 corresponds to a graph where circulating osteocalcin in serum extracted from Wistar rats at the beginning and end of the 90-day experimental period 5 months after the ovariectomy may be observed. Osteocalcin was measured by means of an ELISA test (Enzyme-Linked ImmunoSorbent Assay) from the serum of 8 healthy Wistar rats, 8 ovariectomized Wistar rats treated with intraperitoneal injection of physiological saline solution for 90 days and 8 osteoporotic ovariectomized Wistar rats treated with any of the peptides object of this invention derived from the Cementum-derived attachment protein (HACD1/CAP): AVIFM (SEQ ID NO: 1) (FIG. 14A) or from Cementum Protein 1 (CEMP1): MGTSSTDSQQAQHRRCSTSN (SEQ ID NO: 2) (FIG. 14B); at a concentration of 20-50 μg/Kg of weight dissolved in physiological saline solution and injected intraperitoneally daily, for 90 days 5 months after the ovariectomy.

As may be seen in the graph, levels of serum osteocalcin are increased by 174% as compared to the levels of osteocalcin found in the serum of healthy control rats. However, after 90 days, at the end of treatment of osteoporotic rats with the vehicle used in this pharmaceutical formulation which was physiologic saline solution, serum osteocalcin levels increase up to 205%, while osteoporotic rats treated with any of the peptides object of this invention show a 61% reduction when compared to the values of osteocalcin in rats without osteoporotic treatment with any of the peptides. This indicates that the administration of any of the peptides object of this invention promotes a decrease in the serum osteocalcin levels when compared with rats without osteoporotic treatment thus suggesting that the peptides promote bone remineralization.

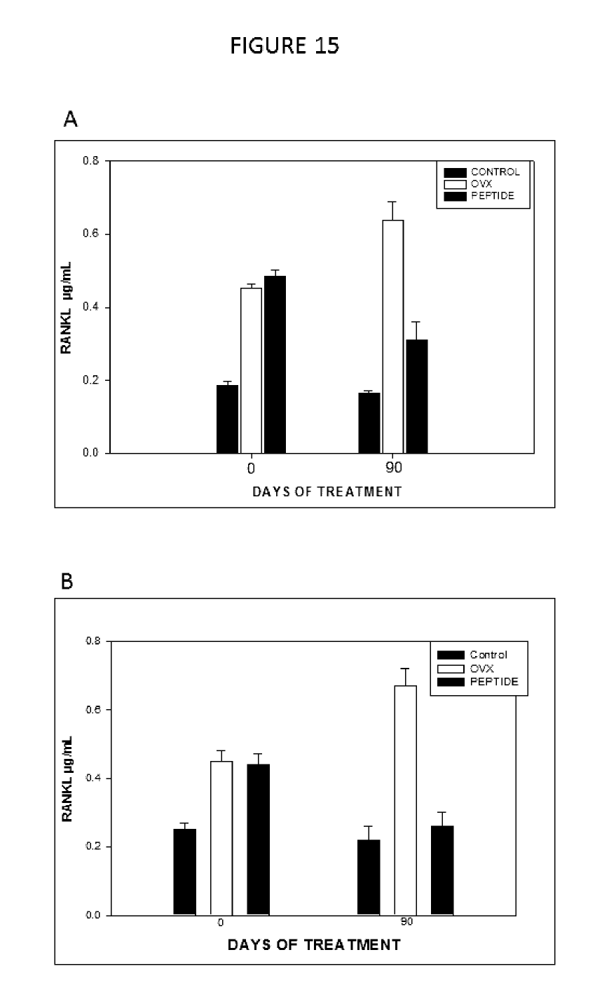

FIG. 15 corresponds to a graph where circulating receptor-activator for nuclear factor κ B ligand (RANKL) may be observed in serum extracted from Wistar rats at the beginning and end of the 90-day experimental period 5 months after the ovariectomy. RANKL levels were determined by means of an ELISA assay (Enzyme-Linked ImmunoSorbent Assay) from the serum of 8 healthy Wistar rats, 8 ovariectomized Wistar rats treated with intraperitoneal injection of physiological saline solution during 90 days and 8 osteoporotic ovariectomized Wistar rats treated with any of the peptides object of this invention derived from the Cementum-derived Attachment Protein (HACD1/CAP): AVIFM (SEQ ID NO: 1) (FIG. 15A) or from Cementum Protein 1 (CEMP1): MGTSSTDSQQAQHRRCSTSN (SEQ ID NO: 2) (FIG. 15B) at a concentration of 20-50 μg/Kg of weight dissolved in physiological saline solution and injected intraperitoneally daily for 90 days 5 months after the ovariectomy.

As shown in the graph, RANKL levels in osteoporotic rats at the beginning of treatment are increased by 155% compared to healthy controls. Ninety days after treatment with the peptides object of this invention an increase of 305% of RANKL in serum of osteoporotic rats compared with healthy controls is observed. RANKL levels in osteoporotic Wistar rats treated with any of the peptides object of this invention show a decrease of 53% compared to the levels of RANKL found in rats osteoporotic that received as treatment the intraperitoneal injection of the pharmaceutical formulations consisting of physiological saline solution for 90 days. These results indicate that treatment with any of the peptides object of this invention inhibit the process of bone resorption.

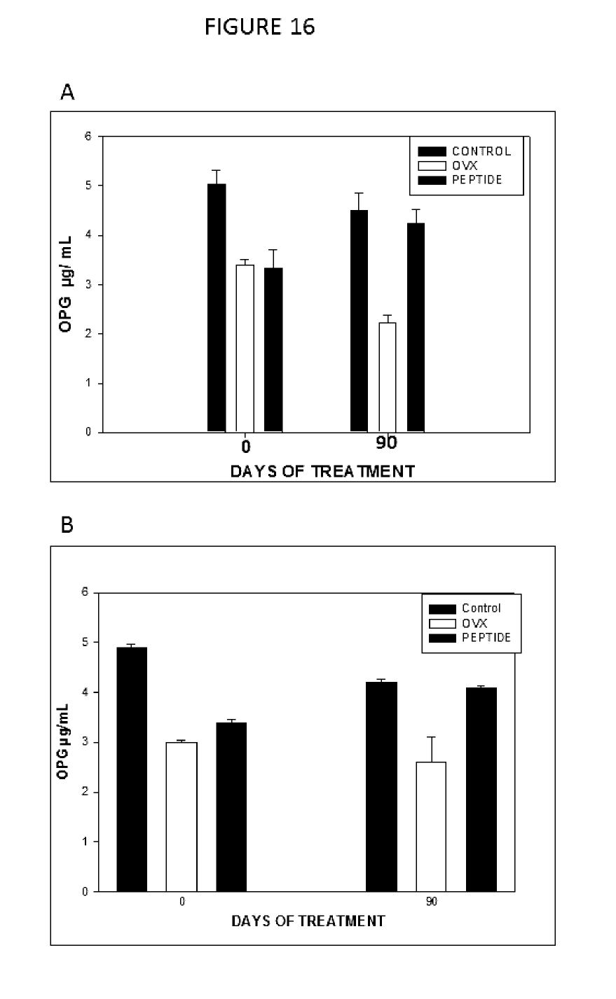

FIG. 16 corresponds to a graph where the level of circulating osteoprotegerin (OPG) in serum extracted from Wistar rats at the beginning and end of the 90-day experimental period, 5 months after ovariectomy, may be observed. Osteoprotegerin levels were determined by means of an ELISA assay (Enzyme-Linked ImmunoSorbent Assay) from the serum of 8 healthy Wistar rats, 8 ovariectomized Wistar rats treated with intraperitoneal injection of physiological saline solution for 90 days and 8 osteoporotic ovariectomized Wistar rats treated with any of the peptides object of this invention derived from the Cementum-derived attachment protein (HACD1/CAP): AVIFM (SEQ ID NO: 1) (FIG. 16A) or from Cementum Protein 1 (CEMP1): MGTSSTDSQQAQHRRCSTSN (SEQ ID NO: 2) (FIG. 16B); at a concentration of 20-50 μg/Kg of weight dissolved in physiological saline solution and injected intraperitoneally daily for 90 days 5 months after the ovariectomy. As shown in the graphs in FIG. 16, osteoprotegerin serum levels in osteoporotic rats at the beginning of treatment decreased by 35% compared to healthy controls. Ninety days after treatment with the peptides object of this invention an increase in serum circulating osteoprotegerin levels are similar to healthy controls with only a 2% difference. The results indicate that the administration of any of the peptides object of this invention increase the levels of serum osteoprotegerin, which is interpreted as a regulatory role in bone metabolism, inhibiting the resorption process and promoting bone remineralization which is reflected in an increase in bone mineral density and in the mechanical properties of bone tissue.

The results of the performance of the peptide of the invention show that for the osteoporotic control rat, the absence of treatment does not prevent bone loss nor the presence of medullary empty spaces in the epiphyses of femur, or prevents bone resorption as seen in the 5^{th} lumbar vertebra accompanied by a reduction in the thickness of the cortical bone that surrounds or covers the vertebral body. Likewise, and as a consequence, the biochemical markers involved in bone apposition and resorption present in the serum suggest that alkaline phosphatase and osteocalcin showed higher values than normal as a result of an increase in bone resorption. Other markers such as osteoprotegerin and RANKL show decreased values with respect to normal values as a result of an increase in bone resorption. The formulation of the peptide AVIFM (SEQ ID NO: 1) or the peptide MGTSSTDSQQAQHRRCSTSN (SEQ ID NO: 2) at a concentration of 20-50 μg/kg of body weight in combination with the vehicle consisting of physiological saline solution shows full medullar spaces without evidence of osteoporotic spaces and with normal bone microarchitecture. Biochemical markers of bone resorption and apposition present in serum show values similar to those obtained from healthy Wistar rats. The tissues histologically examined and shown in this invention do not exhibit any difference with the ones of healthy controls or those obtained from Wistar rats treated with any of the peptides.

To determine the bio distribution of the peptides object of this invention, they were marked using technetium in the form of pertechnetate (TcO₄_{^{−}}). In order to perform the marking, a working solution which contained: 40 ug of the peptides object of this invention, 50 ul of tartrate (2 mg/ml of acetate buffer pH5), 10 μl of SnCl₂ (2 mg/ml 0.1 N HCl), 50 μl of gentisic acid (1 mg/ml) and 50 μl of 99m Tc—Na TCO₄_{^{−}} (5 mCi) was prepared. Male Wistar rats of 250 g of weight which were at a temperature of 22° C. with a photoperiod of 12 hours and relative humidity at 50% were used. Food and water was ad libitum. To assess bio distribution, groups of three rats were formed and injected intraperitoneally with 40 μl of the marked peptide. They were sacrificed by an overdose of anesthetic at 30’, 1, 3, 6 and 24 hours. Blood samples from the heart, lung, liver, spleen, kidneys, stomach, small intestine, large intestine, muscle, bone, thyroid, brain and tooth were collected. These organs were weighed on an analytical balance and subsequently placed in a NaI(TI) solid scintillation counter to detect the activity of each one of the organs. With this procedure, the percentage of the injected dose per gram of tissue (% ID/g) was obtained.

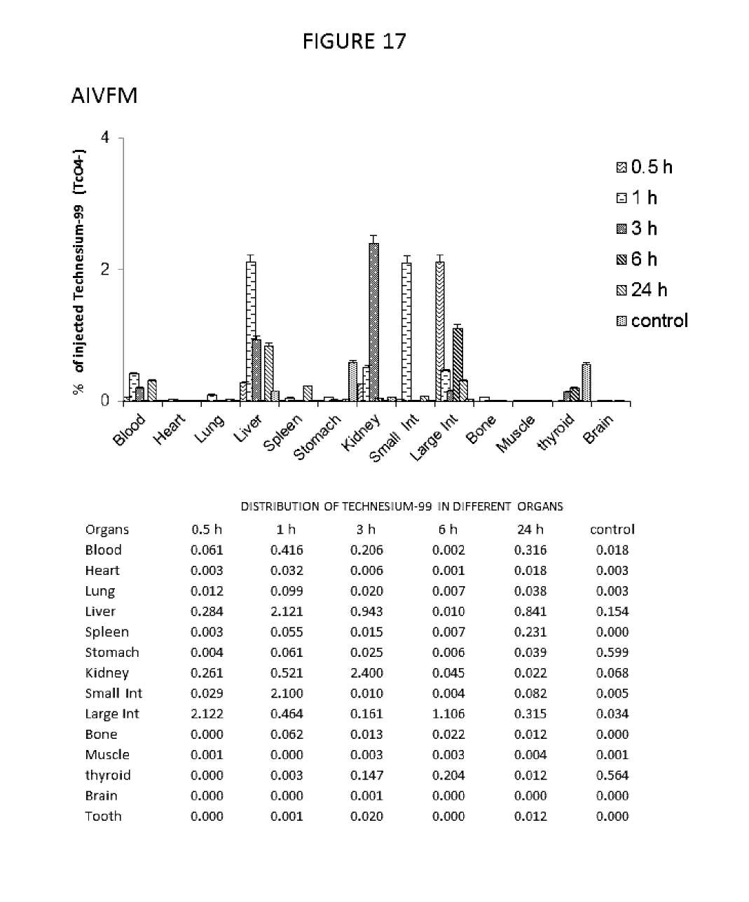

The results indicate that the AVIFM (SEQ ID NO: 1) peptide presents the maximum value of 2.1% in the large intestine at 30′ decreasing its values to 0.3% in 24 hours. One hour after the peptide was administered the organs that showed a greater presence of the peptide with a percentage of 2.1% were the liver and small intestine, decreasing to 0.8 and 0.08% of remnant peptide respectively at 24 hours. In the kidney it reaches its maximum value of 2.4% at 3 hours after peptide injection and decreases to 0.02 percent at 24 hours after the administration. This is illustrated in FIG. 17, graph (A) and in the bio distribution table of this peptide at the different times that were analyzed (B).

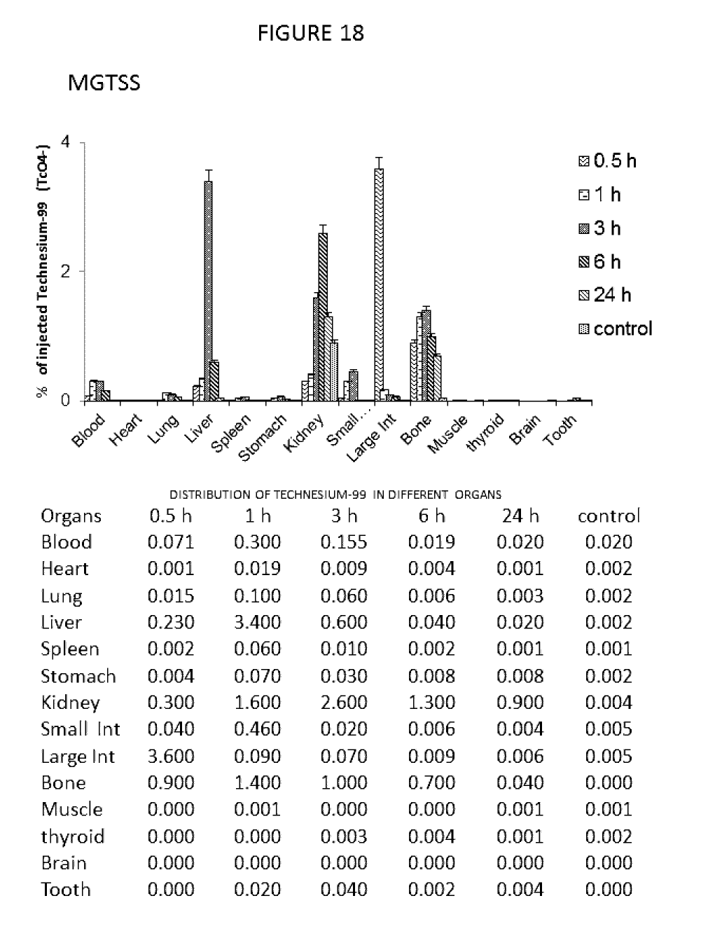

The results indicate that at 30′ the peptide MGTSSTDSQQAQHRRCSTSN (SEQ ID NO: 2) reaches a maximum level of 3.6% in the large intestine only to decrease its levels to 0.006% after 24 hours. In the bone, it is present in a 1.3% and 1.4% as maximum values at 30′ and one hour respectively, decreasing after 24 hours to 0.4%. In the liver, the percentage of remnant peptide reaches 3.4% one hour after the peptide injected dropping to 0.2% in 24 hours. In the kidney percentages of 1.6, 2.6 and 1.3 are present at 1, 3 and 6 hours respectively, with the presence of the peptide decreasing after 24 hours to a 0.9%. Therefore, these are the organs where the highest percentage of remaining peptide was determined. This is illustrated in FIG. 18, graph (A) and in the table with the percentages of peptide presence in the different organs examined (B).

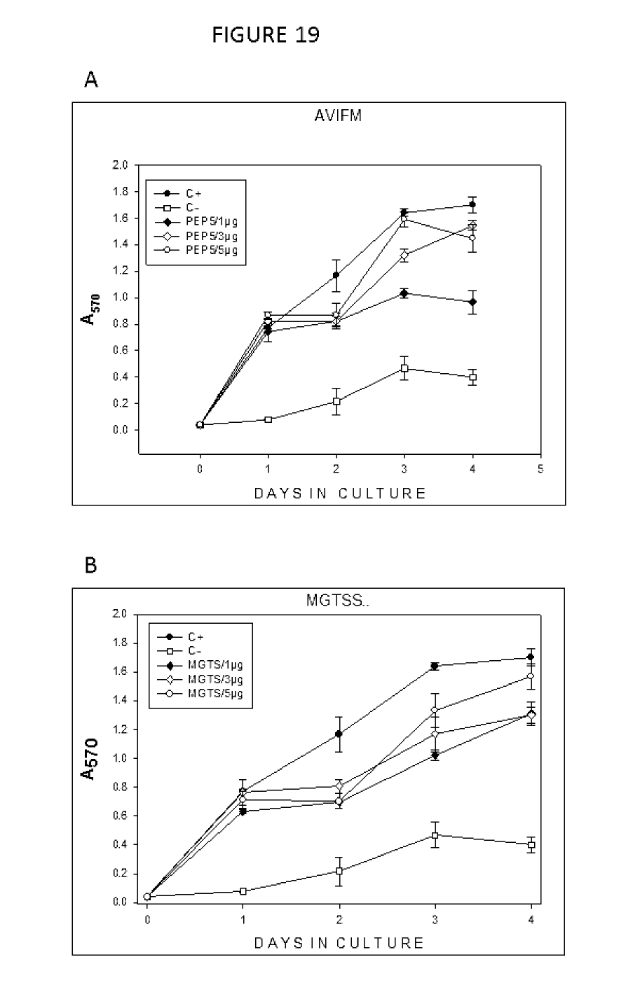

The cytotoxic effect and the effect on cell proliferation was assessed by means of the MTT colorimetric assay (tetrazolium) [3-(4,5-dimethylthazol-2-yl)-2,5-diphenyl tetrazoliumbromide], during 1, 2, 3 and 4 days. Human Osteoblasts derived from alveolar bone were planted at a density of 1×10⁴ in plates of 96 wells. The cells were treated with the peptides (independently) for 4 days with concentrations of 1, 3 and 5 μg/ml of each of the peptides. The medium was replaced daily, and the peptides administered in the same way. The controls used were medium with fetal bovine serum at 10% (positive control) and medium with fetal bovine serum at 0.5% (negative control.) the peptides were administered at the various concentrations contained in the medium at 0.5%. At the end of each day of treatment, 10 μL of MTT (3-(4,5-dimethylthiazol-2-yl)-2,5-diphenyltetrazolium bromide) solution (5 mg/mL) was added to the wells and incubated for 3 hours. At the end the supernatant was removed and extraction was carried out by means of DMSO for 1 hour at 37° C. The optical density of the supernatant was read in a reader for ELISA at 570 nm. The optical density reflects the number of living cells present in the culture. The results of FIGS. 19 A and B show that a 5 μg administration of both peptides used independently promote cell proliferation. The results indicate that at 4 days, the peptide AVIFM (SEQ ID NO: 1) at a concentration of 5 μg/ml promotes an 82% proliferation with regard to the positive control (10% fetal bovine serum) (A). The peptide MGTSSTDSQQAQHRRCSTSN (SEQ ID NO: 2) at a 5 μg/ml concentration promotes cell proliferation to a 92% with respect to the positive control (10% fetal bovine serum) (B). These results indicate that the peptides object of this invention promote cell proliferation and are not cytotoxic to human osteoblasts.

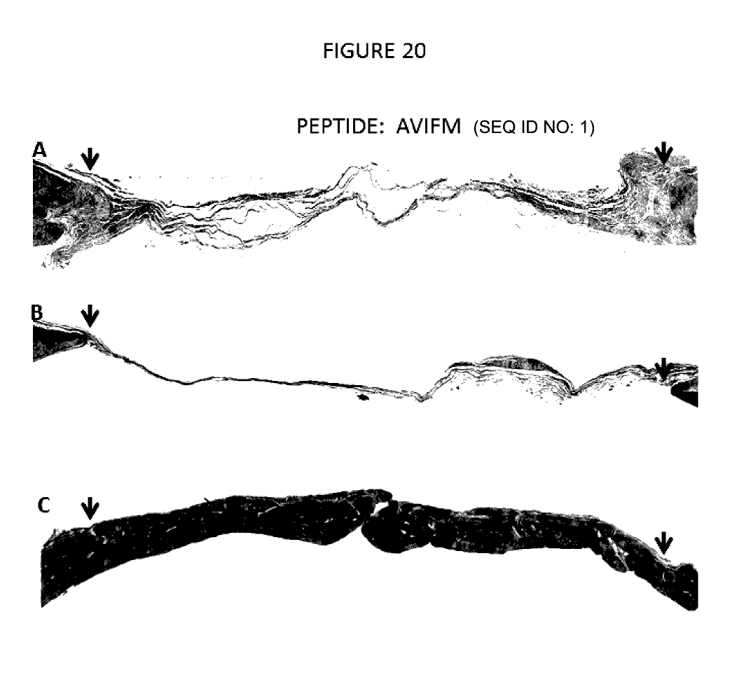

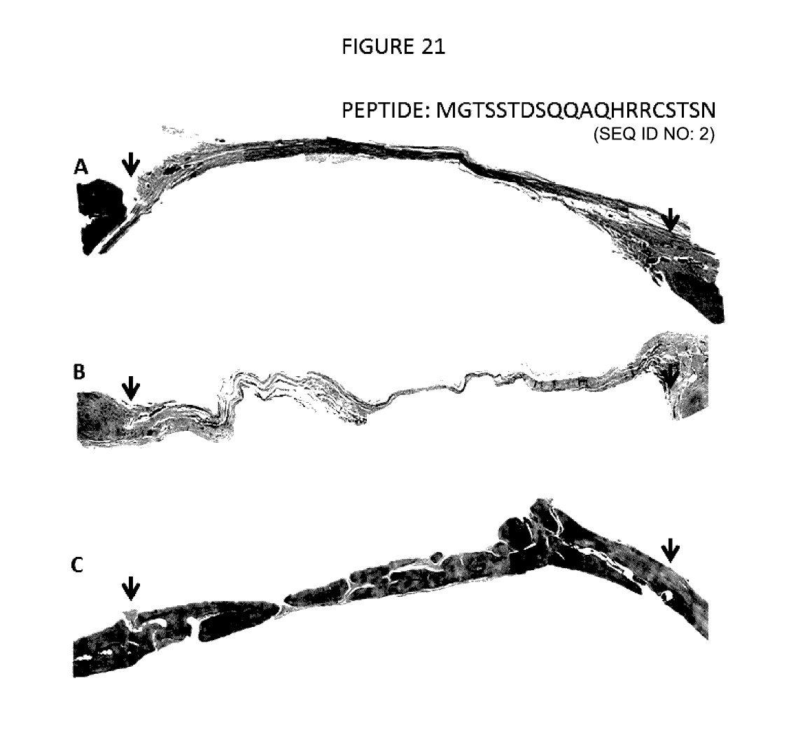

In FIGS. 20 and 21 electron micrographs of the 5-μm thickness histological section are presented and show the calvarial critical size defect in Wistar rats. Letter (A) corresponds to the group that received no treatment and after 16 weeks, it only shows a bridge of fibrous connective tissue with no bone formation or signs of mineralized tissue deposits. Letter (B) corresponds to a section, after 16 weeks, of the control group treated only with the scaffold (Wistar rat) where the critical size defect was covered with a three-dimensional scaffold made of gelatin (gelfoam sponge). There is no evidence of bone formation or indications of mineralized tissue, only the presence of a bridge of fibrous tissue connecting the ends of the defect. Letter (C) corresponds to a section after 16 weeks of the groups that received the formulation of the three-dimensional scaffold (gelatin sponge) plus 20-50 μg of the peptide AVIFM (SEQ ID NO: 1) (FIG. 20) or 20-50 μg of the peptide MGTSSTDSQQAQHRRCSTSN (SEQ ID NO: 2) (FIG. 21), left in situ in the calvarial critical size defect in Wistar rats. It can be seen that 100% of the defect has been filled with material with histomorphologic features of bone tissue with normal characteristics.

Once the peptides of the invention were assessed comparing them with the recombinant proteins and peptides commonly used for therapeutic bone repair and/or regeneration such as parathyroid hormone (PTH) protein related to the parathyroid hormone (PTHrP), peptide amphiphile arrays, peptides derived fromBMP7 (BFPO) and BMP2, the biologically active peptides object of this invention also exhibit the following advantages:

1) They have a similar function to a polypeptide (either natural or recombinant protein) and the active sites of this short chain of amino acids (peptide) may be completely exposed and can be joined to its corresponding receiver at cell surface to achieve better bioactivity. Our experimental results clearly show that any of the peptides described in this invention and to a concentration of 20-50 μg promote the deposition of calcium phosphate salts in the form of hydroxyapatite and promote the self-assembly of these crystals during the in vitro process of mineralization (see experimental results in FIGS. 1 and 2).
2) They are small peptides since their composition is less than 100 amino acids. Small peptides composed of less than 50 amino acids can easily be synthesized artificially. However, synthesis of peptides is difficult and therefore, they are expressed by means of genetic engineering. Compared to the production of recombinant proteins, small peptides exhibit a smaller structure and are more easily produced in larger scales. Therefore, the economic impact on patients who suffer from osteopenia and osteoporosis or who have suffered bone mass loss that requires regeneration is significantly reduced due to the low cost of the peptide production.
3) Due to the relatively small molecular mass of the peptides, therapeutic formulation is more abundant as the peptide may be dissolved in a solution and injected subcutaneously or placed in situ.

The novel features that are considered characteristic of the present invention are determined with particularity in the annexed claims. However, the invention itself, both by its structural organization, jointly with other objects and advantages of the same, will be better understood in the following statements that define the object of the invention and its scope: The present invention provides a peptide that consists of the last five amino acids of the carboxyl terminal of the Cementum-derived attachment protein (HACD1/CAP) with the sequence AVIFM: SEQ ID NO: 1, with nucleating of hydroxyapatite crystals, osteogenic and osteinductive properties.

The present invention provides a peptide derived from the amino terminal of the Cementum protein 1 (CEMP1) that consists of 20 amino acids with the sequence MGTSSTDSQQAQHRRCSTSN: SEQ ID NO: 2, with nucleating of hydroxyapatite crystals, osteogenic and osteinductive properties.

Both peptides possess intrinsic biological activity to increase bone mineral density, restore bone microarchitecture, promote osteogenesis, inhibit bone resorption, increase bone mineral density and therefore act as an effective method for restoring the bone's physical-mechanical characteristics and be clinically effective for the treatment of osteoporosis.

It is yet another object of the present invention to provide a pharmaceutical composition comprising any of the peptide sequences at concentration of 20-50 μg/Kg of weight, combined with a vehicle, rusher or pharmaceutically acceptable excipient, and that are useful in the treatment of osteopenia, osteoporosis and bone regeneration in in vivo systems.

The formulation or pharmaceutical composition of the invention is basically characterized because it includes the following components:

(a) the peptide derived from the Cementum-derived attachment protein (HACD1/CAP) and composed of 5 amino acids: AVIFM (SEQ ID NO: 1),
b) and/or the peptide derived from the Cementum Protein 1 (CEMP1) which consists of 20 amino acids: MGTSSTDSQQAQHRRCSTSN (SEQ ID NO: 2).

Part of the modalities of the invention relate to the pharmaceutical formulations whose active ingredient is the peptide AVIFM (SEQ ID NO: 1) or the peptide MGTSSTDSQQAQHRRCSTSN (SEQ ID NO: 2) that are in combination with a vehicle or an acceptable pharmaceutically excipient including, for example, a pH 7.4 physiological saline solution or a phosphate-buffered solution with a 7.4 pH. This formulation meets the activity profile necessary to counteract the stated problems, but other formulations may be used

It is yet another object of the present invention to provide a pharmaceutical composition comprising the peptide AVIFM (SEQ ID NO: 1) or the peptide MGTSSTDSQQAQHRRCSTSN (SEQ ID NO: 2) that are in combination with a synthetic or natural three-dimensional scaffold which may be a polymer of collagen, elastin, fibrin, hyaluronic acid, chitosan/chitin or alginate, for example it may also be a scaffold formed by a sponge composed primarily of gelatin and which are useful for bone repair and/or regeneration in in vivo systems.

They are forms of the invention the use of any of the peptides alone or in combination to promote the deposition of calcium phosphate salts in the form of hydroxyapatite or in an in vitro system in grafts packed in a suitable medium, or in an in vivo system, i.e. in vertebrates in general but particularly in mammals and more specifically, in humans who suffer from beginnings of osteoporosis (osteopenia) or already suffering from osteoporosis which produces decrease in bone mineral density, bone loss, decrease of the mechanical resistance to fracture and alterations in the protein levels associated with bone metabolism. This is the reason why the pharmaceutical formulations of this invention, as will be explained in more detail later, is able to reverse all these alterations at any stage of the disease and serve as an effective treatment for osteoporosis in living beings.

The peptides object of the invention can be used in humans and vertebrate animals to treat any condition where it is required to promote osteogenesis, inhibit bone resorption, increase bone mineral density, for osteoporosis treatment and bone regeneration in vivo. Also, due to the biological properties that the peptides possess and have demonstrated experimentally in vitro and in vivo they can be used for regeneration of other mineralized tissues such as enamel and dentin as well as therapeutic agents for prevention of bone diseases such as osteopenia, osteoporosis, osteoarthritis and to prevent and treat the fractures that occur in diseases such as osteogenesis imperfecta.

Among the examples of the peptide's application for the correction of bone defects are: its administration after insertion of autografts or allografts, bone bridges or for bone fusion; bone regeneration is also required for the treatment of periodontitis or its sequelae. It should be mentioned with regard to bone bridges that these are used to eliminate joint movement which is a strategy for dealing with degenerative disorders in column and joints, however in up to 45% of column surgeries errors occur in the spinal fusion, leaving the patient with permanent pain and susceptible to repeating surgeries with unsuccessful results. Thus, the present invention also provides an alternative therapy for this condition.

The peptides can be solubilized, prepared and used to decrease and also inhibit the effects of osteoclastogenesis and osteoclastic activity in diseases in which there are bone fractures. It remains within the scope of the invention the possibility of using the peptides in the dose that the attending physician decides to adjust according to the state of the patient, a preferential dose may be in a range of 20-50 μg/Kg of weight. It remains within the scope of the invention the possibility of using the peptides combined for patient treatment according to the criteria of the attending physician.

Examples of other conditions that the patient may present and be dealt with the peptides of the invention include: Paget's bone disease, cancers that metastasize to bone and induce fracture (i.e. multiple myeloma, breast cancer, some melanomas and cancers that do not metastasize to bone but result in hypercalcemia and loss of the bone matrix (such as squamous cell carcinomas), to mention a few that are within the scope of the invention.

The peptides can be synthesized by conventional methods such as Merrifield technique, Sheppard technique, support or solid-phase (Fmoc), etc., with adequate returns at industrial scale.

Even when they have been illustrated and described certain modalities of the invention, it should be emphasized that many modifications are possible, such changes would not represent a distance from the true extent of the invention. Therefore, the present invention should not be regarded as restricted except as provided in the state of the technique, as well as the scope of the appended claims.

In conclusion the experimental results presented here, with this experimental model, indicate that the peptides object of this invention are capable of promoting bone remineralization and regeneration and have no harmful side effects which ensures their viability as a safe therapeutic method for osteoporosis treatment and bone regeneration in vivo.

The present invention will be better understood from the following examples, which are presented for illustrative purposes only to permit full understanding of the preferred modes of the present invention, without implying that there are no other modalities that can be implemented based on the above detailed description.

### EXAMPLES

### Example 1

### Model of Adult Wistar Rats that Demonstrates the Peptides' Use for Osteoporosis Treatment

Peptides with the sequences AVIFM (SEQ ID NO: 1) and MGTSSTDSQQAQHRRCSTSN (SEQ ID NO: 2) have been artificially synthesized and resuspended in a vehicle consisting in physiological saline solution (pH 7.4) that are inoculated intraperitoneally and/or subcutaneously and used for increasing bone mineral density, restoring bone microarchitecture as well as the physical-mechanical and biological properties in an experimental model of ovariectomized Wistar rats (Rattus norvegicus) with osteoporosis. We used 48 female rats of the Wistar strain, 3 months of age, with a weight of 250±20 gr. The animals were acclimated for 14 days at an ambient temperature of 22° C. with a photoperiod of 12 hours and relative humidity of 50%; food and water were ad libitum. The rats were sedated with acepromazine maleate (Relax 0.5 g/100 ml) at a dose of 20 μg/Kg of weight and as general anesthetic, tiletamine and zolazepam were used at a concentration of 25 mg/kg of weight.

Anesthetic maintenance was performed with constant flow of isoflurane. Once the rats were anesthetized we proceeded to perform the bilateral ovariectomy procedure for what the anesthetized rat was placed in lateral decubitus and shaved from the last rib forming a rectangle of approximately 4×3 cm. This area is disinfected with an electrolyzed super oxidant solution with neutral pH (Veteribac). Then a linear incision of approximately 2 cm up was performed with a scalpel to reach the muscle and expose the abdominal cavity. With extreme care, the cervix was located and identified. The cervix was ligated exactly on its middle portion with 6/0 polyglycolic acid sutures and subsequently the ovaries were eliminated with a scalpel. It was sutured by planes (abdominal muscles and skin) with 4/0 polyglycolic acid sutures and finally, the surgical area was cleaned with hydrogen peroxide. The rats were maintained in vivarium conditions during 5 months with 12 hours of light and 12 hours of darkness, at an average temperature of 22° C. with food and water ad libitum. The control group did not receive any surgical procedure and was kept in identical conditions as that of ovariectomized rats. Once this period concluded, 32 ovariectomized rats were divided into 4 groups of 8 rats each. One group received by intraperitoneal injection the peptide AVIFM (SEQ ID NO: 1) and another group, peptide MGTSSTDSQQAQHRRCSTSN (SEQ ID NO: 2); both were resuspended in physiological saline solution (pH 7.4) at a concentration of 20-50 μg/kg of body weight for 90 days. The control group of osteoporotic rats (16 rats) was treated with physiological saline solution (pH 7.4) for 90 days. The healthy control group (16) rats received no treatment at all. Blood was obtained from all groups at the beginning and end of treatment (90 days). The 5^{th} dorsal vertebra of the rats was analyzed by means of computed micro tomography (μCT) prior to euthanasia with CO₂ on day 90 of treatment. The femur and 5^{th} dorsal vertebrae were obtained, fixed in 10% paraformaldehyde and embedded in paraffin. Five μm-thick histological sections were made and stained with Masson's trichrome stain for analysis under light microscope. The histological examination in FIG. 3C or 4C of femoral epiphyses shows that in the groups that received treatment with the peptide AVIFM (SEQ ID NO: 1) or peptide MGTSSTDSQQAQHRRCSTSN (SEQ ID NO: 2) there is bone marrow between cortical spaces and bone trabeculae forms a network structure similar to the microarchitecture of healthy control group (Panels A and C of FIGS. 7 and 8); unlike osteoporotic rats without treatment that exhibited a decrease in cortical bone thickness and marrow spaces occupied by fatty tissue (Panels B of FIGS. 7 and 8). In addition, this histologic pattern in the transverse and sagittal planes reveals homogenous bone trabeculate and filling of inter-trabecular spaces with bone marrow. This is repeated in the body of the 5^{th} lumbar vertebrae. Both healthy controls and osteoporotic rats that received treatment with the peptides object of the invention show similar characteristics in their tissue microarchitecture. Osteoporotic rats show medullar spaces occupied by fatty tissue and a decrease in bone trabeculate.

In addition to what was described histologically, in the mechanical resistance analysis, as may be observed in FIG. 12, osteoporotic rats treated with any of the peptides object of this invention exhibit values of resistance to compression similar to those of healthy control rats, while osteoporotic rats without treatment show significant differences with respect to healthy controls.

Likewise, the analysis of biochemical markers demonstrates that treatment of osteoporotic rats with any of the peptides results in serum levels similar to healthy controls

### Example 2

### Example 2

### Bone Regeneration that Occurs Due to the Presence of Peptides with the Sequences AVIFM (SEQ ID NO: 1) or MGTSSTDSQQAQHRRCSTSN (SEQ ID NO: 2)

These peptides have been artificially synthesized and then combined with a three-dimensional scaffold made of gelatin (gelatin sponge), thus it was used for regeneration of critical size bone defects in an animal model: two groups of 14 male Wistar rats of approximately 250±20 grams of weight were used. Anesthesia of the rats was achieved through intraperitoneal injection of ketamine. The critical size defect was done by performing a 3 cm incision in the calvarial bone and then lifting a mucoperiostial flap. Subsequently, with a 9 mm trephine, a defect was created in the rat's calvarial bone. In the experimental group the formulation was introduced. It consisted of a 9 mm-diameter, porous, three-dimensional, gelatin-based (Gelfoam) disk containing the peptide at a concentration of 20-50 μg/disk. Previously, the dimensional scaffold (gelatin sponge) was incubated with a concentration of the corresponding peptide at 20-50 μg/disk of gelatin sponge and was vacuum dried. Control animals were treated only with a disk of gelatin sponge and a vehicle for re-suspension of the peptide (phosphate buffer pH 7.4) and another group remained without treatment. Blood supply is ensured at the surgical site. The operation site was sealed with Satin S-100, which consists primarily in gelatin. At the end of 16 weeks, the tissues were fixed with 10% formaldehyde and embedded in paraffin. Histological sections were made of 5 μm thickness and then stained with Masson's trichrome stain for their analysis under light microscope. Histomorphometric analysis was performed in order to determine the amount of mineralized tissue formed in the critical size defect in the calvarial bone of Wistar rats. Histological analyses are shown in FIGS. 20 and 21 for the sequences AVIFM (SEQ ID NO: 1) and MGTSSTDSQQAQHRRCSTSN (SEQ ID NO: 2) which show that in the group that received three-dimensional Gelfoam only, neither osteogenesis nor formation of osteoid material associated with total degradation and complete absorption of the vehicle (gelatin sponge), occur as can be seen in the letter of FIGS. 20 and 21, respectively, as well as in the no-treatment group, as can be seen in (B) of FIGS. 20 and 21, respectively. Whereas in the groups that received the formula consisting of either the peptide AVIFM (SEQ ID NO: 1) or peptide MGTSSTDSQQAQHRRCSTSN (SEQ ID NO: 2) in conjunction with the three-dimensional scaffold of gelatin sponge, a 100% repair was achieved in the filling of the defect (with bone) as is clearly seen in (C) of FIGS. 20 and 21, respectively.

### Example 3

### Example 3

### Manufacture of Three-Dimensional Scaffolds for Pre-Clinical Models

The three-dimensional scaffolds used in this invention are based on a gelatin sponge of commercially called Gelfoam®, which is an absorbable gelatin in sponge presentation. In this invention gelatin disks with a diameter of 9 mm and a thickness of 2 mm were cut. This dimensional gelatin sponge was soaked with the peptide AVIFM (SEQ ID NO: 1) or peptide MGTSSTDSQQAQHRRCSTSN (SEQ ID NO: 2) respectively, previously diluted in sterile saline solution at a concentration of 20-50 μg of either of the peptides object of this invention. Subsequently, the three-dimensional scaffold containing the peptide is desiccated in a desiccator at a 4° C. temperature for 1 hour. The three-dimensional scaffold in disk form was irradiated with ultraviolet light for 24 hours and used immediately to cover the defect of critical size defect in calvarial bone of Wistar rats.

1. An osteogenic peptide selected from the group that consists of: peptide AVIFM identified as SEQ ID NO: 1 and peptide MGTSSTDSQQAQHRRCSTSN identified as SEQ ID NO: 2 and possible variants of the same that maintain their osteogenic activity.

2. A method for promoting osteogenesis, increasing bone mineral density and/or promoting bone regeneration comprising administering the osteogenic peptide of claim 1 to a subject in need thereof.

3. A pharmaceutical composition comprising the osteogenic peptide of claim 1.

4. A method for promoting bone regeneration in vivo in vertebrates comprising administering the osteogenic peptide of claim 1 to the vertebrate in need thereof.

5. The method of claim 4 wherein the vertebrates is humans.

6. The method according to claim 2, wherein osteogenesis implies in situ regulation and promotion of deposition of calcium and phosphorus salts, nucleation of hydroxyapatite crystals and their self-assembly during the mineralization process in conditions where bone neo-formation is required.

7. A method for preventing or treating osteoporosis comprising administering the osteogenic peptide according to claim 1 to a subject in need thereof.

8. A pharmaceutical composition comprising as active principle at least one of the peptides selected from the group consisting of: peptide AVIFM identified as SEQ ID NO: 1 and peptide MGTSSTDSQQAQHRRCSTSN identified as SEQ ID NO: 2, and a pharmaceutically acceptable carrier, excipient or diluent, including a synthetic or natural polymer pharmaceutically acceptable.

9. The composition according to claim 8, wherein the excipient or diluent of the peptide AVIFM identified as SEQ ID NO: 1 or peptide MGTSSTDSQQAQHRRCSTSN identified as SEQ ID NO: 2, is a pH 7.4 phosphate-buffered solution and the polymer is made of collagen, elastin, fibrin, hyaluronic acid, chitosan/chitin or alginate.

10. The composition according to claim 8, wherein the peptide AVIFM identified as SEQ ID NO: 1 or the peptide MGTSSTDSQQAQHRRCSTSN identified as SEQ ID NO: 2, may be administered preferentially at a concentration of 20 μg/Kg of weight for osteoporosis treatment and bone regeneration.

11. The composition according to claim 7, wherein the peptide AVIFM identified as SEQ ID NO: 1 or the peptide MGTSSTDSQQAQHRRCSTSN identified as SEQ ID NO: 2, may be administered preferentially at a concentration of 20 μg/Kg of weight for osteoporosis treatment and bone regeneration.


SEQUENCE LISTING (4 sequences)

SEQ ID NO: 1 - protein, 5 aa, Homo sapiens
AVIFM

SEQ ID NO: 2 - protein, 20 aa, Homo sapiens
MGTSSTDSQQAQHRRCSTSN

SEQ ID NO: 3 - protein, 11 aa, Homo sapiens
IVHCLIGIVPT

SEQ ID NO: 4 - protein, 15 aa, Homo sapiens
VSFPSCCFSIAVIFM